(19) United States
(12) Patent Application Publication
(10) Pub. No.: US 2006/0166276 A1
(43) Pub. Date: Jul. 27, 2006

(54) Method of diagnosis and agents useful for same

(75) Inventors: Ian R. Doyle, Marino (AU); Carmine De Pasquale, Prospect (AU); Andrew D. Bersten, Seacliff (AU); Leonard Arnolda, East Fremantle (AU)

Correspondence Address: SEED INTELLECTUAL PROPERTY LAW GROUP PLLC, 701 FIFTH AVE, SUITE 6300, SEATTLE, WA, 98104-7092, US

(73) Assignee: Lung Health Diagnostics Pty Ltd, Bedford Park (AU)

(21) Appl. No.: 11/215,839

(22) Filed: Aug. 29, 2005

Related U.S. Application Data
(63) Continuation of application No. PCT/AU04/00252, filed on Feb. 27, 2004.
(63) Continuation-in-part of application No. 09/486,703, filed on Jun. 27, 2000.
(60) Provisional application No. 60/450,201, filed on Feb. 27, 2003.

(30) Foreign Application Priority Data
Aug. 4, 1998 (AU) P05062
Sep. 5, 1997 (AU) P08999

(51) Int. Cl. G01N 33/53; G01N 33/00

(52) U.S. Cl. 435/7.1; 436/86

(57) ABSTRACT

The present invention relates generally to a method of diagnosing, predicting or monitoring the development or progress of heart failure in a mammal and, more particularly, to a method of diagnosing, predicting or monitoring the development or progress of congestive heart failure in a mammal. The present invention contemplates a method for detecting heart failure by screening for the systemic presence of pulmonary surfactant protein in a subject. The present invention further provides a method for diagnosing or monitoring conditions associated with or characterised by the onset of heart failure, in particular congestive heart failure. Also provided are diagnostic agents useful for detecting one or more surfactant proteins.

### FIELD OF THE INVENTION

The present invention relates generally to a method of diagnosing, predicting or monitoring the development or progress of heart failure in a mammal and, more particularly, to a method of diagnosing, predicting or monitoring the development or progress of congestive heart failure in a mammal. The present invention contemplates a method for detecting heart failure by screening for the systemic presence of pulmonary surfactant protein in a subject. The present invention further provides a method for diagnosing or monitoring conditions associated with or characterised by the onset of heart failure, in particular congestive heart failure. Also provided are diagnostic agents useful for detecting one or more surfactant proteins.

### BACKGROUND OF THE INVENTION

Bibliographic details of the publications referred to by author in this specification are collected alphabetically at the end of the description.

The reference to any prior art in this specification is not, and should not be taken as, an acknowledgement or any form of suggestion that that prior art forms part of the common general knowledge in Australia.

Heart failure, in particular congestive heart failure, is a disease for which no ready “cure” exists. Accordingly, due to the large proportion of the Western population who are affected, it is associated with a high mortality rate (Senni M, Tribouilloy C M Rodeheffer R J et al., Circulation 1998;98:2282-9; Chin M H., Am J Med 1999;107:634-6). For example, nearly 5 million Americans are currently living with this condition, with 550,000 new cases diagnosed each year. Congestive heart failure affects people of all ages, from children to young adults to the middle-aged to senior citizens. However, it is more common among older people. Therefore, as the older population grows over the next few decades, so will the number of people living with congestive heart failure. Further, apart from the high mortality rate associated with congestive heart failure, the long term management of congestive heart failure patients is characterised by episodes of heart failure decompensation (Givertz M M, Collucci W S, Braunwald E., Heart Disease. A textbook if cardiovascular medicine. Philadelphia: WB Saunders Co; 2001:534-57), which result in recurrent (often lengthy) hospital admissions and an enormous financial burden on all Western societies (McMurray J, Hart W., Eur Heart J 1993;14:133; Krum H., Med J Aust 1997;167:61-2), consistently accounting for approximately 70% of total health care costs in these nations (Krum et al., 1997, supra).

The term “heart failure” describes the condition where the heart is unable to pump blood at its normal level. Usually, this is due to the heart having been weakened over time by underlying problems such as clogged arteries, high blood pressure, a defect in the heart's muscular walls or valves or some other medical condition.

Heart failure usually manifests as a gradually worsening chronic disease. Utilising the diagnostic tests currently available, the heart has usually been losing pumping capacity for a significant period of time. The difficulty in conclusively diagnosing the onset of heart failure at an early stage in a patient is attributable, at least in part, to the fact that the onset of heart disease can be associated with a significant period of over-compensation by the heart, resulting in a period of asymptomatic heart failure. Specifically, a “failing” heart compensates by:
(i) Enlarging—when the heart chamber enlarges, it stretches more and can contract more strongly, thereby pumping more blood;
(ii) Developing more muscle mass—the increase in muscle mass occurs because of an increase in size of the contracting cells of the heart, allowing the heart to, at least initially, pump more strongly; and
(iii) Pumping faster thereby helping to increase the output of the heart.

The body may further try to compensate in other ways. For example, blood vessels will narrow to maintain blood pressure, thereby attempting to compensate for the heart's loss of power. The body will divert blood away from less important tissues and organs to maintain flow to the most vital organs, being the heart and brains. These temporary measures only mask the problem of heart failure, but they do not solve it. This tends to explain why some people may not become aware of their condition until many years after the heart begins to decline. Ultimately, the heart and body are unable to maintain the illusion of normal physiology and the patient will begin to experience the fatigue, breathing problems or other more serious symptoms that are generally associated with a heart condition and prompt a trip to the doctor.

Due to the invasiveness and expense of the diagnostic tests currently utilised to diagnose heart failure (eg. echocardiography, radionuclide ventriculography (multiple-gated acquisition scanning), angiography (catheterization) or electrocardiogram (EKG or ECG)), there can be either patient reluctance or physician reluctance to order such tests in the absence of one or more risk factor indicators (eg. obesity or diabetes) or actual symptomatic evidence (eg. shortness of breath). Since not all sufferers of heart failure exhibit one or more of the well known risk factors, this can often mean that heart failure is not detected until it has become severe and/or chronic (sometimes not even until the first heart attack has occurred) thereby leading to an increase in the incidence of mortality and a significant burden to the health system since late stage diagnosis usually involves more expensive and interventionist monitoring and treatment, as opposed to the simpler lifestyle changes which may suffice if diagnosis occurred at an early stage.

Accordingly, there is a need to develop simple, highly sensitive, accurate and cost-effective diagnostic tests for heart failure which would enable and encourage their routine application even in the absence of the existence of symptoms or risk factors in an individual. Early stage diagnosis would significantly decrease current burdens on the health care systems of most Western nations. Further, a simple yet accurate test would also reduce the requirement for ongoing invasive and expensive currently available diagnostic tests for those experiencing chronic heart failure, thereby providing a two fold benefit to the patient, doctor and health system.

Raised pulmonary microvascular pressure (Pₘᵥ) challenges the strength of the fragile alveolocapillary barrier. In congestive heart failure, chronic Pₘᵥ elevation induces adaptive structural changes, which serve to thicken the alveolocapillary barrier (Heard, B. E., Path, F. C., Steiner, R. E., Herdan, A., Gleason, D. Br J Radiol 41:161-171, 1968; Kay, J. M., Edwards, F. R., J. Pathol. 111:239-245, 1973; Kuroki Y, Tsutahara S, Shijubo N et al., Am Rev Respir Dis 1993; 147:723-9; Townsley, M. I., Fu, Z., Mathieu-Costello, O., West, J. B., Circ Res 77:317-325, 1995; Davies, S. W., Gailey, J., Keegan, J., Balcon, R., Rudd, R. M., Lipkin, D. L., Am Heart J 124:137-142, 1992). This provides protection from further high vascular pressure damage (Townsley et al., 1995, supra; Davies et al., 1992, supra) to the alveolocapillary membrane. Specifically, type I alveolar epithelial cells are replaced with progenitor cuboidal cells in subjects when Pₘᵥ is chronically elevated (Kay et al., 1973, supra; Kuroki et al., 1993, supra). This, coupled with the proliferation of interstitial fibrous tissue ((Kay et al., 1973, supra; Kuroki et al., 1993, supra), will change and attempt to strength the normal architecture of the alveolocapillary barrier.

In work leading up to the present invention it has been surprisingly determined that despite the thickening of the alveolocapillary membrane which occurs with the increase in Pₘᵥ associated with the onset of heart failure, an increase occurs in the permeability, to pulmonary surfactant proteins, of the alveolocapillary membrane. These findings are particularly surprising in light of the results of studies directed to analysis of altered fluid flux, the movement of proteins across the membrane of the lung and the vulnerability of the lungs to hydrostatic damage. Movement of transferrin has been used to study alveolocapillary barrier permeability in congestive heart failure, with conflicting results (Guazzi, J., Clin Sci 98:633-641, 2000; Kaplan, J. D., Calandrino, F. S., Schuster, D. P., Am Rev Respir Dis 143:150-154, 1991; Townsley et al., supra). Townsley and co-workers found no change in the pulmonary microvascular osmotic reflection coefficient of protein in dogs after 7 weeks of pacing-induced congestive heart failure, while Huang and co-workers demonstrated a reduction in vascular protein permeability in an aortic banding model of congestive heart failure (Huang, W., Kingsbury, M. P., Turner, M. A., Donnelly, J. L., Flores, N. A., Sheridan, D. J., Cardiovascular Res 49:207-217, 2001). Further, these analyses have focussed on unidirectional protein movement, unlike the present study which demonstrated the novel concept of bi-directional protein movement (through surfactant protein leakage). That is, not just that protein can move into the lungs in congestive heart failure, but that protein can move out of the lungs.

These findings have now facilitated the development of an assay directed to diagnosing and/or monitoring heart failure based on analysing systemic pulmonary surfactant levels. These levels provide an extremely sensitive diagnostic marker of heart failure, in particular congestive heart failure, or any other condition characterised or otherwise associated with the onset of congestive heart failure.

### SUMMARY OF THE INVENTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

One aspect of the present invention relates to a method for detecting the onset or a predisposition to the onset of heart failure in a mammal, said method comprising screening for the modulation of the systemic levels of pulmonary surfactant in said mammal.

Another aspect of the present invention relates to a method for detecting the onset or a predisposition to the onset of heart failure in a mammal, said method comprising screening for the level of pulmonary surfactant in a body fluid from said mammal wherein an increase in the level of pulmonary surfactant is indicative of heart failure.

Still another aspect of the present invention relates to a method for detecting the onset or a predisposition to the onset of acute or chronic heart failure in a mammal, said method comprising screening for the level of pulmonary surfactant in a body fluid from said mammal wherein an increase in the level of pulmonary surfactant is indicative of the onset of acute or chronic heart failure.

Yet another aspect of the present invention provides a method for detecting the onset or a predisposition to the onset of acute or chronic heart failure in a mammal, said method comprising screening for the level of pulmonary surfactant in a sample of blood from said mammal wherein an increase in the level of pulmonary surfactant is indicative of the onset of acute or chronic heart failure.

In yet still another aspect present invention provides a method for detecting the onset or a predisposition to the onset of acute or chronic heart failure in a mammal, said method comprising screening for the level of one or more of SP-A, SP-B, SP-C and/or SP-D in a sample of blood from said mammal wherein an increase in the level of said SP-A, SP-B, SP-C and/or SP-D is indicative of the onset of acute or chronic heart failure.

Still yet another aspect of the present invention relates to a method for monitoring heart failure in a mammal, said method comprising screening for the modulation of systemic levels of pulmonary surfactant in said mammal.

Still another aspect provides a method for monitoring the progression of heart failure in a mammal, said method comprising screening for the modulation of systemic levels of pulmonary surfactant in said mammal wherein the maintenance of or an increase in the level of pulmonary surfactant relative to a previously obtained surfactant level result in said mammal is indicative of the maintenance or worsening of said heart failure.

Yet still another aspect provides a method for monitoring the progression of heart failure in a mammal, said method comprising screening for the modulation of systemic levels of pulmonary surfactant in said mammal wherein a decrease in the level of pulmonary surfactant relative to a previously obtained surfactant level result in said mammal is indicative of an improvement in said heart failure.

In yet another further aspect the present invention provides a method for assessing the severity of heart failure in a mammal, said method comprising quantitatively screening for the level of pulmonary surfactant in a body fluid from said mammal wherein the degree of increase of said level of pulmonary surfactant is indicative of the severity of said heart failure.

Another aspect of the present invention provides a diagnostic kit for assaying serum samples comprising in compartmental form a first compartment adapted to contain an agent for detecting pulmonary surfactant and a second compartment adapted to contain reagents useful for facilitating the detection by the agent in the first compartment. Further compartments may also be included, for example, to receive a biological sample. The agent may be an antibody or other suitable detecting molecule.

### BRIEF DESCRIPTION OF THE DRAWINGS

FIG. 1 is a graphical representation of the differences in circulating ANP and NT-proBNP levels in CHF patients from controls, and across NYHA class. Data are bar graphs of mean±SEM A) ANP and B) NT-proBNP in n=53 CHF patients (cross hatched bars) and n=19 age-matched controls (open bars). CHF patients are divided into n=19 NYHA class II, n=22 class III and n=14 class IV patients.

A) ANP was elevated above the normal range (100 pg/ml, dotted line) in the CHF patients and changed with NYHA classification (*p<0.01), being markedly elevated in NYHA class IV patients.

B) NT-pro BNP was elevated in CHF, and was elevated in the NYHA class II CHF patient subgroup over the control group, ^{†}p<0.05. NT-proBNP increased sequentially within the CHF group as NYHA classification worsened, ^{‡}p<0.001.

FIG. 2 is a graphical representation of the differences in circulating surfactant protein-A and -B levels in CHF patients from controls, and across NYHA class. Data are bar graphs of mean±SEM A) SP-A and B) SP-B in n=53 CHF patients (cross hatched bars) and n=19 age-matched controls (open bars) as per FIG. 1.

A) SP-A was elevated in CHF patients compared to controls, and was elevated in the class II CHF patient subgroup over the control group (*p<0.001). However, within the CHF group SP-A did not increase significantly as NYHA classification worsened (p=0.3).

B) Surfactant protein-B was elevated in CHF patients compared to controls, and was elevated in the class II CHF patient subgroup over the control group (^{†}p<0.01). Within the CHF group SP-B increased sequentially as NYHA classification worsened (*p<0.001).

FIG. 3 is a graphical representation of the relationship between plasma surfactant protein-B and freedom from CHF hospitalization. Data are n=53 CHF patients divided into tertiles of plasma surfactant protein-B levels (3395, 4509) (33^{rd} and 66^{th} percentile). Kaplan Meier curves depict freedom from CHF hospitalization after 18 months follow up. Higher plasma surfactant protein-B was associated with higher rates of CHF hospitalization, p<0.001.

FIG. 4 is a graphical representation of the change in NT-proBNP with CHF decompensation and treatment. Data are circulating NT-proBNP levels in A) n=21 CHF patients at the previous stable clinic visit (stable), and the clinic visit were loop-diuretic dosage was increased (CHF decompensation), and B) n=32 CHF patients at the clinic visit were loop-diuretic dosage was increased (CHF decompensation) and the follow up clinic visit after treatment (follow up). Diamonds represent median values.

1A) There was an increase in plasma NT-proBNP at the time of CHF decompensation (*p<0.001).

1B) There was a decrease in plasma NT-proBNP following treatment of CHF decompensation*.

FIG. 5 is a graphical representation of the change in surfactant protein-B with CHF decompensation and treatment. Data are circulating SP-B levels in patient groups A) and B) as per FIG. 1. Diamonds represent median values.

2A) There was an increase in plasma SP-B at the time of CHF decompensation (*p<0.001).

2B) There was a decrease in plasma SP-B following treatment of CHF decompensation*.

FIG. 6 is a graphical representation of the change in atrial natriuretic peptide with exercise. Data is circulating ANP level pre and post-exercise, n=20 subjects, closed squares represent median values. Circulating ANP increased post exercise, *p<0.001.

FIG. 7 is a graphical representation of the change in surfactant protein-B with exercise. Data is circulating SP-B level pre and post-exercise, n=20 subjects, closed squares represent median values. Circulating SP-B did not change following exercise.

FIG. 8 is a graphical representation of the change in atrial natriuretic peptide with exercise, split for presence of myocardial ischemia on EST. Data is circulating ANP levels pre and post-exercise, in A) n=10 subjects without evidence of exercise induced myocardial ischemia on EST and, B) n=10 subjects with evidence of exercise induced myocardial ischemia on EST, closed squares represent median values.

A) Circulating ANP increased post negative EST, *p<0.05.

B) Circulating ATP increased post positive EST, ^{†}p<0.01.

FIG. 9 is a graphical representation of the absolute change in ANP and SP-B based on results of exercise stress test. Data is difference in pre and post-exercise circulating ANP and SP-B levels in cohort with negative (n=01), and cohort with positive (n=10) EST for myocardial ischemia. (negative EST: ANP, cross hatched bars; SP-B, clear bars) (positive EST: ANP, checkered bars; SP-B, closed bars)

Circulating ANP increased post exercise in both the negative and positive EST groups with a greater increase in the positive EST group, *p<0.05, ^{†}p<0.01. Circulating SP-B did not change in the negative EST group, but did increase post exercise in the positive EST group, *p<0.05.

FIG. 10 is a graphical representation of the change in surfactant protein-B with exercise, split for presence of myocardial ischemia on EST. Data is circulating SP-B level pre and post-exercise in A) n=10 subjects without evidence of exercise induced myocardial ischemia on EST and, B) n=10 subjects with evidence of exercise induced myocardial ischemia on EST, closed squares represent median values.

A) Circulating SP-B did not change post negative EST.

B) Circulating SP-B increased post positive EST, *p<0.05.

FIG. 11 is a depiction of ascending aortic snare technique.

(A) Snare line is placed around the ascending aorta, when snare line is pulled through the funneled catheter at the interscapular space (B) the snare tightens around the ascending aorta and an acute pressure load is placed on the left ventricle.

FIG. 12 is a graphical representation of changes in hemodynamic parameters with aortic snare constriction. Data are n=6 group II rats with moderate aortic snare constriction (closed squares, dotted line) and n=6 group III rats with severe aortic constriction (at T5) (closed circles, solid line). Graphs are XY Plots of mean±SEM A) Left ventricular end-diastolic pressure (LVEDP), B) Left ventricular systolic pressure (LVSP) and C) mean arterial blood pressure (BP) against time (minutes).

A) LVEDP increased from T0 in groups II and III at T5, *p<0.05. In group III LVEDP increased further at T20, ^{†}p<0.05.

B) LVSP increased from T0 in groups II and III at T5, *p<0.05, and then did not change significantly.

C) BP decreased from T5 to T20 in groups II and III, *p<0.05, with a more striking reduction in group III.

FIG. 13 is a graphical representation of the Changes in arterial blood gas parameters with aortic snare constriction. Data are as per FIG. 11. Graphs are XY Plots of mean±SEM A) Partial pressure of carbon dioxide in arterial blood (PaCO₂), and B) Partial pressure of oxygen in arterial blood (PaO₂) against time (minutes).

A) PaCO₂ increased over the three sampling times in group II, ^{†}p<0.05.

B) PaO₂ decreased over the three sampling times in both groups II and III, ^{†}p<0.05, with a more striking reduction in group III.

FIG. 14 is a graphical representation of the changes in tissue and lavage radiolabel compartmentalization with aortic snare constriction. Bar graphs of mean±SEM tissue % volume A) ⁵¹Cr-RBC, C) ¹²⁵I-albumin and E) ^{99m}Tc-DTPA, and mean±SEM lavage % volume B) ⁵¹Cr-RBC, D) 1²⁵1-albumin and F) ^{99m}Tc-DTPA in the three study groups: group I (controls) (open bars), n=7; group II (moderate aortic snare constriction) (cross hatched bars), n=6; group III, (severe aortic snare constriction) (closed bars), n=6.

A) Tissue ⁵¹Cr-RBC % volume trended towards a reduction as aortic constriction increased, p=0.10.

B) Lavage ⁵¹Cr-RBC % volume increased as aortic constriction increased, *p<0.01, and was higher in group III compared to controls.

C) Tissue ¹²⁵I-albumin % volume increased as aortic constriction increased, ^{†}p<0.05, and was higher in group III compared to controls.

D) Lavage ¹²⁵I-albumin % volume increased as aortic constriction increased, *p<0.01, and was higher in group III compared to controls.

E) Tissue ^{99m}Tc-DTPA % volume increased as aortic constriction increased, *p<0.01, and was higher in group III compared to controls.

F) Lavage ^{99m}Tc-DTPA % volume increased as aortic constriction increased, *p<0.01, and was higher in both groups II and III compared to controls.

FIG. 15 is a graphical representation of the Changes in circulating surfactant protein-B levels with aortic snare constriction. Data are A) group I rats, controls, B) group II rats with moderate aortic snare constriction and C) group III rats with severe aortic constriction (at T5). Graphs are XY Plots of mean±SEM circulating SP-B levels against time (minutes).

There was no difference in circulating SP-B between the three groups at T0.

A) There was no change in circulating SP-B with time, in the controls (T0 to T20)

B) Following moderate aortic snare constriction there was a change in circulating SP-B, p<0.01, with levels at T5 elevated from baseline (T0)*, and remaining elevated at T20, ^{†}p<0.05.

C) Following moderate aortic snare constriction, and further constriction at T5, there was a trend towards a change in circulating SP-B, p=0.01. As in group II there was an increase in circulating SP-B at T5 from baseline (T0)^{†}. However, following further aortic constriction circulating SP-B levels trended towards a reduction, p=0.17, and at T20 circulating SP-B was no longer elevated from baseline levels (T0), p=0.15.

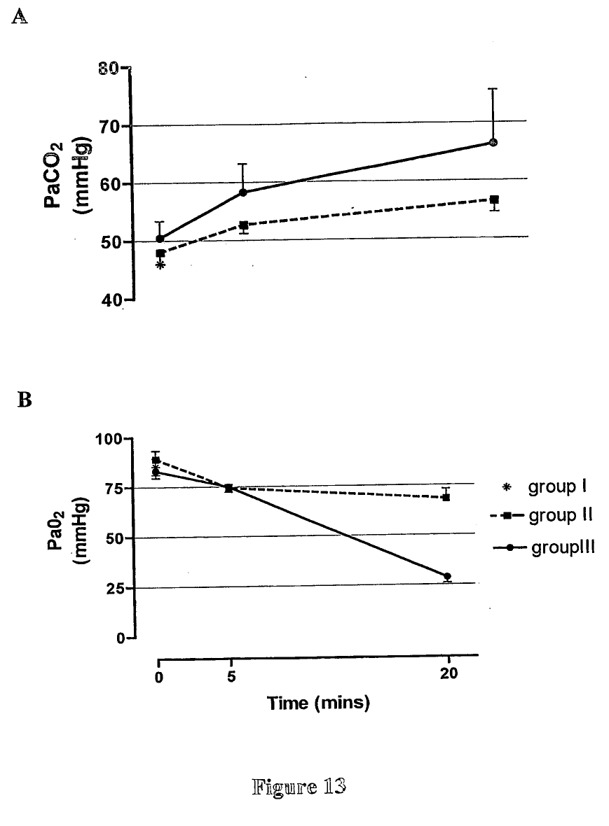

FIG. 16 is a graphical representation of the changes in lavage surfactant protein-B levels with aortic snare constriction. Bar graphs of mean±SEM lavage SP-B, data as per FIG. 13. There was no change in lavage SP-B between the three study groups, p=0.24.

FIG. 17 is a graphical representation of the changes in lung weight with myocardial infarct size. Bar graphs of mean±SEM A) Right upper lobe wet-lung weight per body weight (mg/g), B) Right upper lobe dry-lung weight per body weight (mg/g) and C) wet-to-dry lung weight ratio in the three study groups: controls; 0% LV infarction, n=15, (open bars), moderate infarct group; (25-45% LV infarction), n=17, (cross hatched bars) and large infarct group; (>46% LV infarction), n=7, (closed bars).

A) Wet-lung weight per body weight increased with infarct size, *p<0.001, and was higher in the large infarct group compared to controls*.

B) Wet-lung weight per body weight increased with infarct size*, and was higher in the large infarct group compared to controls*.

C) Wet-to-dry lung weight ratio was unchanged by infarct size.

FIG. 18 is a graphical representation of the changes in derived values of lung water with myocardial infarct size. Bar graphs of mean±SEM A) Intravascular lung water (IVLW) (ml/right upper lobe), B) Extravascular lung water (EVLW) (ml/right upper lobe) and Extravascular lung water per bloodless dry lung weight (ml/g) in the three study groups as per FIG. 17.

A) IVLW decreased with infarct size, *p<0.05, and was lower in the large infarct group compared to controls*.

B) EVLW increased with infarct size, ^{†}p<0.001, and was higher in the large infarct group compared to controls^{†}.

C) EVLW/DLW increased with infarct size^{†}, and was higher in the large infarct group compared to controls.

FIG. 19 is a graphical representation of the changes in tissue and lavage radiolabel compartmentalization with myocardial infarct size. Bar graphs of mean±SEM tissue A) ⁵¹Cr-RBC, C) ¹²⁵I-albumin and E) ^{99m}Tc-DTPA, and mean±SEM lavage percent volume B) ⁵¹Cr-RBC, D) 125I-albumin and F) ^{99m}Tc-DTPA in the three study groups as per FIG. 17.

A) Tissue ⁵¹Cr-RBC fell as infarct size increased, *p<0.05, and was lower in the large infarct group compared to controls*.

B) Lavage percent volume ⁵¹Cr-RBC was unchanged by infarct size.

C) Tissue ¹²⁵I-albumin increased with infarct size, ^{†}p<0.001, and was higher in the large infarct group compared to controls^{†}.

D) Lavage percent volume ¹²⁵I-albumin increased with infarct size^{†}, and was higher in the large infarct group, ^{‡}p<0.01 compared to controls.

E) Tissue ^{99m}Tc-DTPA increased with infarct size^{‡}, and was higher in the large infarct group compared to controls^{‡}.

F) Lavage percent volume ^{99m}Tc-DTPA increased with infarct size*, and was higher in both the moderate infarct group* and the large infarct group, ^{§}p<0.025 compared to controls.

FIG. 20 is a graphical representation of the changes in plasma and lavage surfactant protein-B with infarct size. Bar graphs of mean±SEM A) plasma, B) lavage and C) plasma/lavage ratio SP-B in the three study groups as per FIG. 17.

A) Plasma SP-B increased with infarct size, *p<0.001, and was higher in both the moderate infarct group, ^{†}p<0.05, and the large infarct group* compared to controls.

B) Lavage SP-B increased with infarct size^{†}, and was higher in the large infarct group compared to controls^{†}.

C) Plasma/lavage SP-B ratio increased with infarct size, ^{‡}p<0.01, and was higher in both the moderate infarct group^{†}, and the large infarct group, ^{§}p<0.025 compared to controls.

FIG. 21 is a graphical representation of the changes in lavage fluid evidence of inflammation with infarct size. Scatter plots and median values of lavage fluid A) cell count, and B) percent neutrophils, and C) bar graph of mean±SEM lavage fluid myeloperoxidase activity in the three study groups as per FIG. 17.

A) Lavage cell count increased with infarct size, *p<0.01, and was higher in the large infarct group compared to controls*.

B) Lavage fluid percent neutrophils increased with infarct size*, and was higher in the large infarct group compared to controls*.

C) Lavage fluid myeloperoxidase activity increased with infarct size, ^{†}p<0.05, and was higher in the large infarct group compared to controls^{†}.

FIG. 22 is a graphical representation of the change in clinical pulmonary edema score with time. Data are mean±SEM; n=28 patients with APE. The clinical pulmonary edema score was maximally elevated in all patients at presentation (day 0) and fell sequentially from days 0 to 7. *p<0.001.

FIG. 23 is a graphical representation of the change in plasma surfactant protein-A with time after acute cardiogenic pulmonary edema (APE). Data are mean±SEM; n=28 patients with APE; n=13 normal controls. Plasma surfactant protein-A was elevated at presentation (day 0) compared with age-matched normal controls (*p±0.02). Over the five sampling times there was a change in plasma surfactant protein-A (†p±0.02), with peak levels at day 3 (‡p±0.001) compared with presentation.

FIG. 24 is a graphical representation of the change in plasma surfactant protein-B with time after acute cardiogenic pulmonary edema (APE). Plasma surfactant protein-B was elevated at presentation (day 0) compared with age-matched normal controls (*p=0.01). Over the five sampling times, there was a change in plasma SP-B (†p=0.001), with peak levels at day 3 (‡p=0.008) compared with presentation. Plasma surfactant protein-B then fell to below presentation levels by day 14 (§p=0.001).

FIG. 25 is a graphical representation of the change in plasma tumor necrosis factor-α with time after acute cardiogenic pulmonary edema (APE). Plasma tumor necrosis factor-α elevated at presentation (day 0) compared with age-matched normal controls (*p=0.02). Over the five sampling times, there was a change in plasma tumor necrosis factor-α (*p<0.001). Tumor necrosis factor-α levels peaked at day 1 (‡p=0.02) and remained elevated from presentation levels at day 3 (§p=0.04).

FIG. 26 is a graphical representation of the relationship between peak (day 1) plasma tumor necrosis factor (TNF)-α and chest radiograph extravascular lung H₂O (EVLW) score.

FIG. 27 is a graphical representation of the results of case study 1. Change in plasma surfactant protein-B. Plasma surfactant protein-B is plotted against time, measured in days from the acute cardiogenic pulmonary edema (APE) episode. The first sample (day −9) represents the day of admission to the hospital with unstable angina. †Day −5 was the day of acute myocardial infarction, and onset of APE is marked by the dotted line at day 0. Plasma surfactant protein-B was stable until the myocardial infarction, at which time it gradually increased until the onset of APE (day 0), when there was a marked elevation in plasma surfactant protein-B, peaking at day 3, before falling.

FIG. 28 is a graphical representation of the results of case study 2. Change in plasma surfactant protein-B (SP-B). ‡,§Plasma SP-B is plotted against time (days) from the acute cardiogenic pulmonary edema (APE) episodes (vertical dotted lines). †Mean±SEM baseline plasma SP-B over three clinic visits in preceding 6 months, during which congestive heart failure was stable. *At admission to hospital with worsening heart failure, 6 hrs before first APE episode, surfactant protein-B was elevated compared with baseline. ‡Onset of APE was associated with a further increase in plasma SP-B, peaking at day 3. After falling initially, SP-B remained at presentation (day 0) levels until at least day 12. §At the onset of the second episode of APE (day 14/0′), plasma SP-B again increased, peaking at day 2′ and falling to below presentation levels from day 4′.

### DETAILED DESCRIPTION OF THE INVENTION

The present invention is predicated, in part, on the surprising determination that congestive heart failure is associated with a form of alveolocapillary membrane damage which facilitates the bi-directional flow of protein. These findings have been made despite the prior art teachings that Pₘᵥ elevation, which occurs in congestive heart failure, results in thickening of the alveolocapillary barrier and, further, that movement of proteins across this barrier has previously been found to be unchanged or, even, reduced. Accordingly, the correlation between serum pulmonary surfactant levels and diagnosis of the development or severity of congestive heart failure has now facilitated the development of a simple yet highly sensitive diagnostic assay.

Accordingly, one aspect of the present invention relates to a method for detecting the onset or a predisposition to the onset of heart failure in a mammal, said method comprising screening for the modulation of the systemic levels of pulmonary surfactant in said mammal.

More particularly, the present invention relates to a method for detecting the onset or a predisposition to the onset of heart failure in a mammal, said method comprising screening for the level of pulmonary surfactant in a body fluid from said mammal wherein an increase in the level of pulmonary surfactant is indicative of heart failure.

Reference to “heart failure” should be understood as a reference to a condition where the heart is not pumping, or otherwise functioning, as well as a normal heart. Without limiting the present invention to any one theory or mode of action, a heart which is undergoing heart failure has been weakened over time by an underlying problem. In this regard, the onset of heart failure may be naturally occurring (such as the loss of blood-pumping ability that can occur as one ages) or it can be the result of a congenital abnormality or an acquired abnormality. Examples of congenital abnormalities which can cause the onset of heart failure include abnormal heart valves, cardiomyopathy, abnormally formed heart chambers and myocardial disease. Acquired abnormalities include development of coronary artery disease, the consequences of previous myocardial ischeamia, hypertension, myocarditis (eg. due to viral infection), hypertension, toxic myocardial disease, onset of severe lung disease, hyperthyroidism, arrhythmia or dysrrhythmia, heart muscle damage caused by excessive drug or alcohol use or severe anemia. It should also be understood that acute pulmonary cardiogenic oedema is a form of acute heart failure and is therefore distinguished from non-cardiogenic pulmonary oedema. Reference herein to “acute pulmonary oedema” should be understood to correspond to “acute cardiogenic pulmonary oedema”. Preferably, said forms of heart failure include congestive heart failure and, more specifically, acute and chronic heart failure.

It should be understood that there is a wide range of heart failure severity. Mild heart failure may be asymptomatic due to the body's natural compensatory mechanisms which are initially induced. However, as the failure (weakening) of the heart becomes more severe, it will be associated with increased symptomology. In this regard, the severity of heart failure is usually classified according to how severe an individual's symptoms are. The most commonly used classification system is the New York Heart Association Functional Classification which places patients into one of four categories based on the extent to which they are limited during physical activity, as follows:

| Class | Symptoms |
|---|---|
| I | No symptoms and no limitation in ordinary physical activity |
| II | Mild symptoms and slight limitation during ordinary activity. |
| | Comfortable at rest. |
| III | Marked limitation in activity due to symptoms, even during less- |
| | than-ordinary activity. Comfortable only at rest. |
| IV | Severe limitations. Experiences symptoms even while at rest. |

However, common to all levels of severity of heart failure is the increase in systemic levels of pulmonary surfactant. Preferably, the subject heart failure is congestive heart failure and even more preferably acute or chronic heart failure.

According to this preferred embodiment, the present invention relates to a method for detecting the onset or a predisposition to the onset of acute or chronic heart failure in a mammal, said method comprising screening for the level of pulmonary surfactant in a body fluid from said mammal wherein an increase in the level of pulmonary surfactant is indicative of the onset of acute or chronic heart failure.

Still without limiting the present invention to any one theory or mode of action, heart failure can involve the left side of the heart, the right side or both. However, the left side is usually affected first. Each side is made up of two chambers: the atrium, or upper chamber, and the ventricle, or lower chamber. The atrium receives blood into the heart and the ventricle pumps it to the tissues. Heart failure occurs when any of these chambers lose their ability to keep up with normal blood flow.

Left-sided or left-ventricular (LV) heart failure involves the left ventricle (lower chamber) of the heart. Oxygen-rich blood travels from the lungs to the left atrium, then on to the left ventricle, which pumps it to the rest of the body. Because this chamber supplies most of the heart's pumping power, it is larger than the others and essential for normal functioning. If the left ventricle loses its ability to contract (systolic failure), the heart cannot pump with sufficient force to push enough blood into circulation. If it loses its ability to relax (diastolic failure) due to the muscle having become stiff, the heart cannot properly fill with blood during the resting period between each beat. In either case, blood coming into the left chamber from the lungs may “back up”, causing pulmonary edema. Further, as the heart's ability to pump decreases, blood flow slows down, causing fluid to build up in tissues throughout the body (edema).

The right atrium receives the venous blood and pumps it into the lungs to be re-oxygenated. Right-sided or right-ventricular (RV) heart failure usually occurs as a result of left-sided failure. When the left ventricle fails, increased fluid pressure is, in effect, transferred back through the lungs, ultimately damaging the heart's right side. When the right side loses pumping power, blood backs up in the body's veins. This is often associated with swelling in the legs and ankles.

Despite the detailed understanding which has emerged in relation to the mechanics of heart failure and the associated consequences in terms of pulmonary edema, the complexities in relation to the changes and damage to the lungs as a result of this process were not previously fully understood. In fact, teaching existed that although fluid moved across the lung, the changes to the lung structure were not of a type which would facilitate the passage of proteins across the alveolocapillary membrane. In this regard, studies directed to looking at the movement of various proteins indicated the incidence of little or no movement of proteins out of the lung.

Accordingly, it has been determined that movement of pulmonary surfactant proteins out of the lung results in an increase in the systemic levels of these proteins. By “systemic” is meant that the subject pulmonary surfactant is detectable outside the localised area of the lung tissue. Preferably, said pulmonary surfactant is detected in a body fluid. Reference to “body fluid” should be understood to include reference to fluids derived from the body of a mammal such as, but not limited to, blood (including all blood derived components, for example, serum and plasma), urine, tears, bronchial secretions or mucus and fluids which have been introduced into the body of a mammal and subsequently removed such as, for example, the saline solution extracted from the lung following lung lavage. Preferably, the body fluid is blood or urine and even more preferably blood.

The present invention therefore preferably provides a method for detecting the onset or a predisposition to the onset of acute or chronic heart failure in a mammal, said method comprising screening for the level of pulmonary surfactant in a sample of blood from said mammal wherein an increase in the level of pulmonary surfactant is indicative of the onset of acute or chronic heart failure.

The term “mammal” as used herein includes humans, primates, livestock animals (e.g. horses, cattle, sheep, pigs, donkeys), laboratory test animals (e.g. mice, rats, rabbits, guinea pigs), companion animals (e.g. dogs, cats) and captive wild animals (e.g. kangaroos, deer, foxes). Preferably, the mammal is a human or a laboratory test animal. Even more preferably, the mammal is a human.

As detailed hereinbefore, the present invention is directed to diagnosing the onset of heart failure via detection of an increase in systemic levels of pulmonary surfactant. Without limiting the present invention in any way, the gas/liquid interface of the lung is lined with a monomolecular layer comprising phospholipid, neutral lipids and specific proteins (surfactant proteins A, B, D and D, herein referred to as SP-A, -B, -C and -D, respectively). Collectively known as “pulmonary surfactant”, these compounds lower surface tension, decrease the work of breathing, and stabilise the lung by varying surface tension allowing alveoli of different sizes to co-exist.

Pulmonary surfactant phospholipids are synthesised by Alveolar Type II cells where they are stored in distinctive vesicles known as lamellar bodies. In response to a variety of stimuli, in particular physical distortion of the type II cells, the contents of the lamellar bodies are released into the hypophase, where they hydrate to form a 3-D lattice structure known as tubular myelin. The tubular myelin in turn supplies the monomolecular layer at the gas/liquid interface that possesses the biophysical activity.

The components of the monomolecular layer have a defined life and are constantly replaced. The disaturated phospholipids (DSP) are credited with reducing surface tension to the very low values thought to occur at low lung volumes, while cholesterol, the second most abundant pulmonary surfactant lipid, is thought to affect the rate of adsorption and the fluidity of newly released material. The system is extremely dynamic; in rats, dipalmitoylphosphatidylcholine, the main component of mammalian pulmonary surfactant, has a half-life of −85 minutes in the alveolus with as much as 85% taken back into type II cells and reutilised (Nicholas T E. NIPS 1993; 8:12-8).

To date, four proteins, SP-A, -B, -C and -D have been shown to be uniquely associated with mammalian pulmonary surfactant. There is a general consensus that the extremely hydrophobic proteins (SP-B and -C) are functional components of the monomolecular layer, whereas the more hydrophilic protein, SP-A appears to be more involved in pulmonary surfactant homeostasis and host defence, and SP-D is solely involved in host defence.

Accordingly, reference herein to “pulmonary surfactant” should be read as including reference to all forms of pulmonary surfactant and derivatives thereof including but not limited to pulmonary phospholipid, pulmonary neutral lipids and pulmonary surfactant proteins, and includes all subunit molecules including, by way of example, the precursor, preproproteins, proprotein and intermediate forms of SP-B. Examples of pulmonary surfactant proteins include SP-A, -B, -C and -D. Preferably, said pulmonary surfactant is SP-A, -B, -C or -D. Reference herein to “SP-A” “SP-B”, “SP-C” and “SP-D” should be understood to include reference to all forms of these molecules including all precursor, proprotein and intermediate forms thereof.

The present invention therefore preferably provides a method for detecting acute or chronic heart failure in a mammal, said method comprising screening for the level of one or more of SP-A, SP-B, SP-C and/or SP-D in a sample of blood from said mammal wherein an increase in the level of said SP-A, SP-B, SP-C and/or SP-D is indicative of the onset of acute or chronic heart failure.

Preferably, said pulmonary surfactant is SP-B.

Reference to “detecting” heart failure or a condition characterised by heart failure should be understood in its broadest context and includes, inter alia, diagnosing, screening, confirming or otherwise assessing heart failure or a condition characterised by the onset of heart failure. In a particularly preferred embodiment, the method of the present invention is directed to resolving whether or not a patient presenting with symptoms or other non-exclusive indicators of heart failure has developed heart failure or is predisposed to the onset of heart failure. For example, increased levels of BNP is a non-specific indicator of a number of conditions including the onset of heart failure or pulmonary hypertension. Accordingly, application of the method of the present invention to patients exhibiting increased levels of BNP provides a means of resolving which of these patients are suffering from the onset of heart failure and which have undergone the onset of some other condition requiring further investigation. Accordingly, in a most preferred embodiment, the present invention provides a means of resolving the onset or a predisposition to the onset of heart failure in a patient presenting with one or more non-exclusive symptoms or diagnostic indicators of heart failure. By “non-exclusive” is meant that the symptoms or indicator may be associated with conditions other than just heart failure.

Although not intending to limit the invention to any one theory or mode of action, alveolocapillary membrane damage of the type that has now been found to occur in heart failure causes an increase in alveolocapillary permeability. Although immunoreactive SP-A and SP-B are not normally present in appreciable amounts in the systemic circulation, the appearance of additional pulmonary surfactant proteins in the serum of patients with heart failure occurs as the result of changes in alveolocapillary permeability.

The method of the present invention is predicated on the correlation of levels of pulmonary surfactant in individuals with normal levels of molecules. The “normal level” is the level of pulmonary surfactant in a corresponding biological sample of an age-matched individual who has not developed heart failure nor is predisposed to the development of heart failure. As detailed above, it is predicted that the systemic level of pulmonary surfactants in a normal individual will be negligible or non-existent.

Accordingly, the term “modulation” refers to increases and decreases in serum pulmonary surfactant levels relative either to a normal reference level (or normal reference level range) or to an earlier surfactant level result determined from the body fluid of said mammal. A normal reference level is the surfactant level from the body fluid of a mammal or group of mammals which do not have acute or chronic heart failure. In a preferred embodiment, said normal reference level is the level determined from one or more subjects of a relevant cohort to that of the subject being screened by the method of the invention. By “relevant cohort” is meant a cohort characterised by one or more features which are also characteristic of the subject who is the subject of screening. These features include, but are not limited to, age, gender, ethnicity, smoker/non-smoker status, or pulmonary health status. This reference level may be a discrete figure or may be a range of figures. The reference level may vary between individual classes of surfactant molecules. For example, the normal level of SP-A may differ to the normal level of SP-B or a particular SP-B subunit. Preferably, said modulation is an increase in blood pulmonary surfactant levels.

Although the preferred method is to detect an increase in blood pulmonary surfactant levels in order to diagnose the onset or confirm the existence of heart failure, the detection of a decrease in surfactant levels may be desired under certain circumstances. For example, to monitor improvement in alveolocapillary membrane morphology, and therefore heart function, during the course of prophylactic or therapeutic treatment of patients presenting with heart failure or predisposition to the development of heart failure.

This aspect of the present invention also enables one to monitor the progression of a heart failure condition. By “progression” is meant the ongoing nature of a heart failure condition, such as its improvement, maintenance, worsening or a change in the level of its severity.

Accordingly, another aspect of the present invention relates to a method for monitoring the progression of heart failure in a mammal, said method comprising screening for the modulation of systemic levels of pulmonary surfactant in said mammal.

Preferably, said heart failure is acute or chronic heart failure and said pulmonary surfactant is SP-A, SP-B, SP-C and/or SP-D. Even more preferably, said pulmonary surfactant is SP-B and said systemic level is the blood level.

It should be understood that in accordance with this aspect of the present invention, blood surfactant levels will likely be assessed relative to one or more previously obtained blood surfactant results in the patient.

In one preferred embodiment the present invention provides a method for monitoring the progression of heart failure in a mammal, said method comprising screening for the modulation of systemic levels of pulmonary surfactant in said mammal wherein the maintenance of increase in the level of pulmonary surfactant relative to a previously obtained surfactant level result in said mammal is indicative of the maintenance or worsening of said heart failure.

Preferably, said heart failure is acute or chronic heart failure and said pulmonary surfactant is SP-A, SP-B, SP-C and/or SP-D. Even more preferably, said pulmonary surfactant is SP-B and said systemic level is the blood level.

In another preferred embodiment the present invention relates to a method for monitoring the progression of heart failure in a mammal, said method comprising screening for the modulation of systemic levels of pulmonary surfactant in said mammal wherein a decrease in the level of pulmonary surfactant relative to a previously obtained surfactant level result in said mammal is indicative of an improvement in said heart failure.

Preferably, said heart failure is acute or chronic heart failure and said pulmonary surfactant is SP-A, SP-B, SP-C and/or SP-D. Even more preferably, said pulmonary surfactant is SP-B and said systemic level is the blood level.

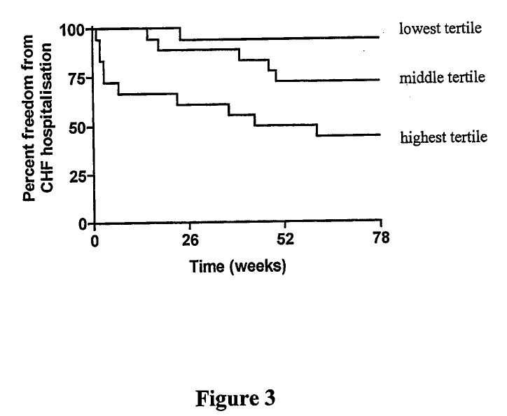

The inventors have still further determined that a correlation exists in relation to the quantitative level of surfactant which is observed in the blood of a patient and the severity of the heart failure from which that patient is suffering. Specifically, the higher the level of surfactant, the more severe the heart failure. Accordingly, the present invention provides a means of both diagnosing and monitoring the existence of heart failure in a qualitative way and also assessing the severity of the heart failure in a patient at a given point in time. In this regard, the severity of heart failure will generally, although not necessarily, be described in terms of its classification under the NYHA system. In one particularly preferred embodiment, it has been determined that SP-B over a defined level or a change (increase) in the level of SP-B is predictive of the requirement for subsequent hospitalisation (see FIG. 3). In the context of the routine analysis of outpatients, this is an extremely valuable tool.

Accordingly, in yet another aspect the present invention provides a method for assessing the severity of heart failure in a mammal, said method comprising quantitatively screening for the level of pulmonary surfactant in a body fluid from said mammal wherein the degree of increase of said level of pulmonary surfactant is indicative of the severity of said heart failure.

Preferably said heart failure is acute or chronic heart failure and said pulmonary surfactant is SP-A, SP-B, SP-C and/or SP-D. Even more preferably, said surfactant is SP-B and said systemic level is the blood level.

Most preferably said SP-B level is predictive of subsequent hospitalisation of the patient.

It should be understood that the ability to assess the severity of heart failure also facilitates the assessment of whether a patient may be predisposed to developing still more severe heart failure. In this context, and in terms of the diagnostic method of the present invention, in general, one may seek to analyse surfactant levels together with one or more other physical parameters—whether they be diagnostic outcomes (eg. stress test results) or even assessment of lifestyle issues.

The method of the present invention has widespread applications including, but not limited to, diagnostic/prognostic analysis of congestive heart failure or the heart failure symptoms or aspects of any condition characterised by the presence of congestive heart failure such as patients with abnormal heart valves or abnormally formed heart chambers, toxic or metabolic myocardial disease, hyperthyroidism, arrhythmia, dysrrhythmia, coronary artery disease, myocardial ischaemia, hypertension, myocarditis, hypertension, severe lung disease or heart muscle damage. The method of the present invention also has application in assessment of the heart health status of any individual irrespective of any perceived predisposition or possibility of having developed heart failure.

Accordingly, another aspect of the present invention is directed to a method of detecting the onset of or a predisposition to the onset of a condition characterised by the presence of heart failure said method comprising screening for the modulation of systemic levels of pulmonary surfactant in said mammal wherein an increase in the level of pulmonary surfactant relative to normal levels is indicative of the onset or predisposition to the onset of said condition.

Yet another aspect of the present invention is directed to a method for monitoring a condition characterised by the presence of heart failure in a mammal said method comprising screening for the modulation of systemic levels of pulmonary surfactant in said mammal wherein maintenance of or an increase in the level of pulmonary surfactant relative to a previously obtained surfactant level result in said mammal is indicative of the maintenance or worsening of said condition.

Still another aspect of the invention is directed to a method for monitoring a condition characterised by the presence of heart failure in a mammal said method comprising screening for the modulation of systemic levels of pulmonary surfactant in said mammal wherein a decrease in the level of pulmonary surfactant relative to a previously obtained surfactant level result in said mammal is indicative of an improvement in said condition.

Preferably said heart failure is acute or chronic heart failure and said pulmonary surfactant is SP-A, SP-B, SP-C and/or SP-D. Even more preferably, said surfactant is SP-B and said systemic level is the blood level.

It should be understood that the screening methodology herein defined may be performed either quantitatively or qualitatively. Although it is likely that quantitative analyses will be preferred since they provide information in relation to both the existence, or not, of heart failure in addition to identifying its severity, the method of the present invention does facilitate qualitative analyses. In particular, since pulmonary surfactants are usually not found in the blood in appreciable amounts, a test directed to assessing the presence or not of a given pulmonary surfactant will provide useful information. It will also provide scope for establishing extremely simple and inexpensive screening procedures.

Screening of pulmonary surfactant levels in the serum of a mammal can be achieved via a number of techniques such as functional tests, enzymatic tests or immunological tests. Functional tests may include detecting SP-A or -B by their ability to affect release or re-uptake of surfactant or by detecting host defence properties. SP-C may be detected by measuring associated palmitates. Immunological tests may include contacting a serum sample with an antibody specific for a pulmonary surfactant (or group of pulmonary surfactants) or its derivatives thereof for a time and under conditions sufficient for an antibody-pulmonary surfactant complex to form, and then detecting said complex.

In one particular preferred method the target surfactant molecules in the serum sample are exposed to a specific antibody which may or may not be labelled with a reporter molecule. Depending on the amount of target and the strength of the reporter molecule signal, a bound target may be detectable by direct labelling with an antibody. Alternatively, a second labelled antibody, specific to the first antibody is exposed to the target-first antibody complex to form a target-first antibody-second antibody tertiary complex. The complex is detected by the signal emitted by the reporter molecule.

By “reporter molecule” as used in the present specification, is meant a molecule which, by its chemical nature, provides an analytically identifiable signal which allows the detection of antigen-bound antibody. Detection may be either qualitative or quantitative. The most commonly used reporter molecules in this type of assay are either enzymes, fluorophores or radionuclide containing molecules (i.e. radioisotopes) and chemiluminescent molecules.

In the case of an enzyme immunoassay, an enzyme is conjugated to the second antibody, generally by means of glutaraldehyde or periodate. As will be readily recognized, however, a wide variety of different conjugation techniques exist, which are readily available to the skilled artisan. Commonly used enzymes include horseradish peroxidase, glucose oxidase, beta-galactosidase and alkaline phosphatase, amongst others. The substrates to be used with the specific enzymes are generally chosen for the production, upon hydrolysis by the corresponding enzyme, of a detectable colour change. Examples of suitable enzymes include alkaline phosphatase and peroxidase. It is also possible to employ fluorogenic substrates, which yield a fluorescent product rather than the chromogenic substrates noted above. In all cases, the enzyme-labelled antibody is added to the first antibody hapten complex, allowed to bind, and then the excess reagent is washed away. A solution containing the appropriate substrate is then added to the complex of antibody-antigen-antibody. The substrate will react with the enzyme linked to the second antibody, giving a qualitative visual signal, which may be further quantitated, usually spectrophotometrically, to give an indication of the amount of hapten which was present in the sample.

Alternatively, fluorescent compounds, such as fluorescein and rhodamine, may be chemically coupled to antibodies without altering their binding capacity. When activated by illumination with light of a particular wavelength, the fluorochrome-labelled antibody adsorbs the light energy, inducing a state to excitability in the molecule, followed by emission of the light at a characteristic colour visually detectable with a light microscope. As in the EIA, the fluorescent labelled antibody is allowed to bind to the first antibody-hapten complex. After washing off the unbound reagent, the remaining tertiary complex is then exposed to the light of the appropriate wavelength the fluorescence observed indicates the presence of the hapten of interest. Immunofluorescence and EIA techniques are both very well established in the art and are particularly preferred for the present method. However, other reporter molecules, such as radioisotope, chemiluminescent or bioluminescent molecules, may also be employed.

The method of the present invention should be understood to include both one off measurements of surfactant levels in a mammal and multiple measurements conducted over a period of time (for example as may be required for the ongoing monitoring of an individual mammal's heart failure status).

Another aspect of the present invention provides a diagnostic kit for assaying biological samples comprising in compartmental form a first compartment adapted to contain an agent for detecting pulmonary surfactant and a second compartment adapted to contain reagents useful for facilitating the detection by the agent in the first compartment. Further compartments may also be included, for example, to receive a biological sample. The agent may be an antibody or other suitable detecting molecule.

Further features of the present invention are more fully described in the following Examples.

### EXAMPLE 1

### Biomarkers of Alveolocapillary Barrier Damage in Chronic Heart Failure

Materials and Methods

Patients and Procedures.

Fifty-three consecutive CHF patients mean±SEM age 66±2 years (18 female, 35 male) from the Flinders Medical Centre Heart Failure Clinic were assessed as outpatients. Assessment comprised of;

1) Dyspnea score (DS). A previously validated questionnaire-based dyspnea score to document subjective dyspnea (Mahler D A, Weinberg D H, Wells C K et al., Chest 1984;85:751-8; Feinstein A R, Fisher M B, Pigeon J G., Am J Cardiol 1989;64:50-5).

2) Left ventricular failure score (LVFS). Objective signs and specific symptoms of left ventricular failure, quantified using a scoring system based on the Framingham criteria for diagnosing decompensated heart failure (Ho 93). Scoring system: chest crepitations (basal=0.5 points, ⅓^{rd}=1, >⅔^{rd}=1.5), third heart sound (present=0.5), orthopnea (possible=0.5, definite=1), paroxysmal nocturnal dyspnea (<2 episodes/week=1, greater than 3 episodes/week=2).

3) A 6-minute walk test (6-MWT).

4) NYHA functional classification of CHF.

5) Venous blood collected for atrial nautriuretic peptide (ANP), NT-proBNP and SP-A and -B assay.

Nineteen age-matched volunteers (69±3 years), free of cardio-respiratory disease, had venous blood sampled for NT-proBNP and SP-A and -B assay to act as a control group.

Specimen Handling and Assays.

Venous blood was collected in EDTA tubes for ANP assay, and lithium heparin tubes for NT-proBNP and, SP-A and -B assay. Blood was centrifuged at 5000 rpm for 5 minutes at 15° C. and the supernatant frozen at −70° C. for random, blinded, batch analysis. ANP was measured by radioimmunoassay (Oliver 88). NT-proBNP was measured using a commercially available electrochemiluminescence sandwich immunoassay (proBNP, ELECSYS 2010, Roche Diagnostics, Mannheim, Germany). SP-A and -B levels were measured using a competitive enzyme-linked immunosorbent assay (ELISA) (Doyle 95, 97).

Statistical Analysis.

Data were analyzed using SPSS for Windows release 10.0. ANP, SP-A and SP-B levels were logarithmically transformed to allow analysis with parametric tests. NT-proBNP levels were not normally distributed following data transformation (Kolmogorov-Smirnov Test) and hence non-parametric data analysis was performed. All data is presented as mean±SEM with statistical significance defined as p<0.05.

SP-A and -B levels in CHF patients and controls were compared using the Student's t test. For normally distributed data differences in measured parameters at the clinic assessment according to NYHA class, were tested using one-way ANOVA, with post hoc comparisons between adjacent NYHA classes with modified Bonferroni correction. Similarly for non-normally distributed data, levels across NYHA classes were compared using the Kruskal-Wallis Test, with between group comparisons using the Mann Whitney U Test with modified Bonferroni correction. Correlations between the measured parameters were assessed using Spearman's Test.

A conditional logistic regression model was used to determine the predictive value of plasma NT-proBNP and SP-B for CHF admission and death.

Results

Of the 53 CHF patients in the study, 17 were NYHA class II, 22 were class III and 14 were class IV.

Difference in Measured Parameters across NYHA Classification.

As expected increasing NYHA functional classification was associated with reduced dyspnea score (lower score worse), increased left ventricular failure score (higher score worse), reduced 6-MWT distance, and increased mortality and rates of CHF hospitalization over an 18 month follow up period. Age also increased as NYHA classification worsened (Table 1).

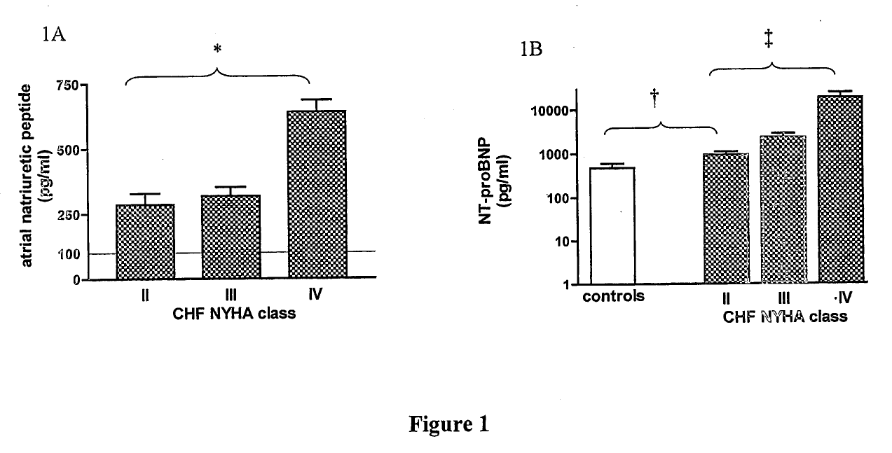

Plasma ANP was elevated in the CHF patients 356±34 pg/ml (normal range<100 pg/ml) (FIG. 1A). Furthermore, plasma ANP changed with NYHA classification in the CHF cohort (FIG. 1A). Plasma NT-proBNP was elevated in the CHF patients compared to the controls, (6765±2033 pg/ml vs. 470±120 pg/ml, p<0.001). Indeed, plasma levels were elevated in the NYHA class II subgroup of patients compared to the controls. Furthermore, levels increased with NYHA classification (FIG. 1B).

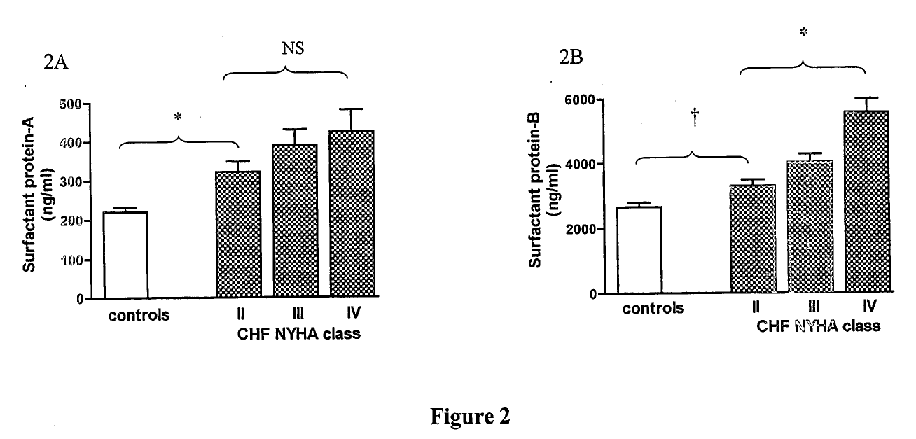

Plasma SP-A was elevated in the CHF patients compared to the controls (377±24 pg/ml vs. 220±112 pg/ml, p<0.001). However, plasma SP-A did not change with NYHA classification (FIG. 2A). Plasma SP-B was also elevated in the CHF patients (4197±200 vs. 2632±196, p<0.001). Again, plasma levels were elevated in the NYHA class II subgroup compared to the controls, and levels increased with NYHA classification (FIG. 2B).

Correlations between Measured Parameters.

As expected the dyspnea score, LV failure score and 6-MWT distances correlated (Table 2). SP-A and -B, and, ANP and NT-proBNP were also related, consistent with their similar release mechanisms. SP-B and NT-proBNP levels were positively correlated. SP-B in particular, correlated with the measured clinical parameters of CHF status (Table 2).

Predictive Value of Biomarkers.

In the 53 CHF patients there were 16 CHF hospital admissions and 9 deaths in the 18-month follow up period. Because of cross correlations between measured parameters, subjects in whom events occurred were matched for NYHA class, age (within 8 years), DS (within 1 point), LVFS (within 1.5 points) and 6-MWT distance (within 30 m) with subjects free of events. In patients subsequently hospitalized for CIF both NT-proBNP and SP-B were elevated (Table 3). Similarly for death, both NT-proBNP and SP-B were elevated (Table 3). On conditional logistic regression analysis plasma SP-B as a continuous variable was independently predictive for CHF hospitalization (Odds Ratio 1.00154, 95% CI 1.00047, 1.00262), (p=0.005), while NT-proBNP was not (p=0.24). Consequently, for each 1000 ng/ml increase in SP-B an excess risk of 4.7-fold was noted (FIG. 3). Due to small numbers of events, mortality conditional logistic regression analysis was not performed.

Subgroup Analysis of CHF Patients Who Died During Follow Up.

Nine patients died during the CHF follow/up period. Six deaths occurred in hospital and were clinically consistent with terminal circulatory/pump failure. The remaining three deaths were sudden and occurred out of hospital without preceding clinical deterioration. There was no difference in age, dyspnea score, LV failure score or circulating SP-A and-B levels between the two causes of death. However, despite the small event numbers, NT-proBNP was significantly higher in the CHF patients who died of circulatory/pump failure compared to those who died suddenly 32968±10934 pg/ml vs. 3874±2793 pg/ml, p=0.035.

### EXAMPLE 2

### Fluctuations in Plasma Biomarker Levels through Congestive Heart Failure Decompensation Episodes and their Treatment

Materials and Methods

Patients and Procedures.

Fifty-three consecutive CHF patients mean±SEM age 66±2 years (18 female, 35 male) from the Flinders Medical Centre Heart Failure Clinic were assessed longitudinally at each clinic visit over an 18 month period. Standardised assessment was as per Example 1 with the addition of body weight and documentation of the decision by the treating cardiologist to increase loop-diuretic dosage (without knowledge of plasma NT-proBNP and SP-B levels).

Specimen Handling and Assays.

Venous blood was collected and stored as per Example 1 and NT-proBNP (Example 1) and SP-B assays were performed in a blinded batch analysis.

Statistical Analysis.

Data were analyzed using SPSS for Windows release 10.0. Non-parametric data analysis was performed for both ordinal and continuous variables. All data is presented as median (25^{th}, 75^{th} percentile), with statistical significance defined as p<0.05. When the treating cardiologist elected to increase the loop-diuretic dosage, the measured parameters were compared to those at the previous clinic visit using the Wilcoxon Signed Ranks Test. When the loop-diuretic dosage was increased the measured parameters at that clinic visit and the follow up visit were compared in the same way.

Results

Change in Measured Parameters at the Time of Increased Loop-Diuretic Dose.

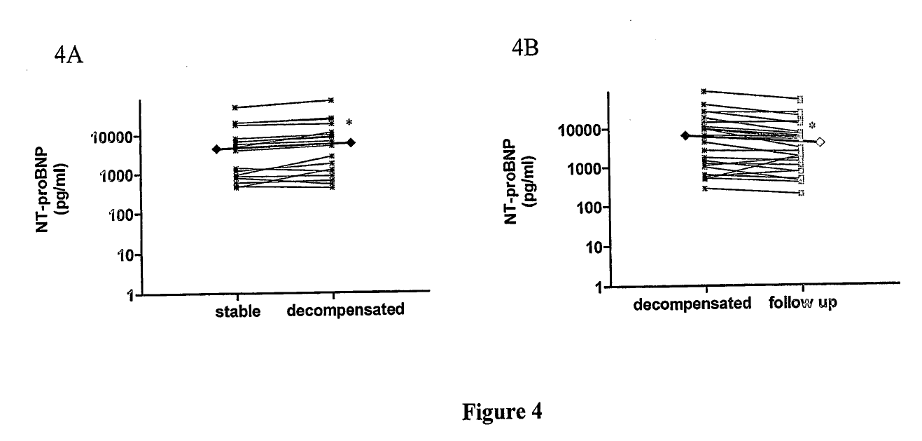

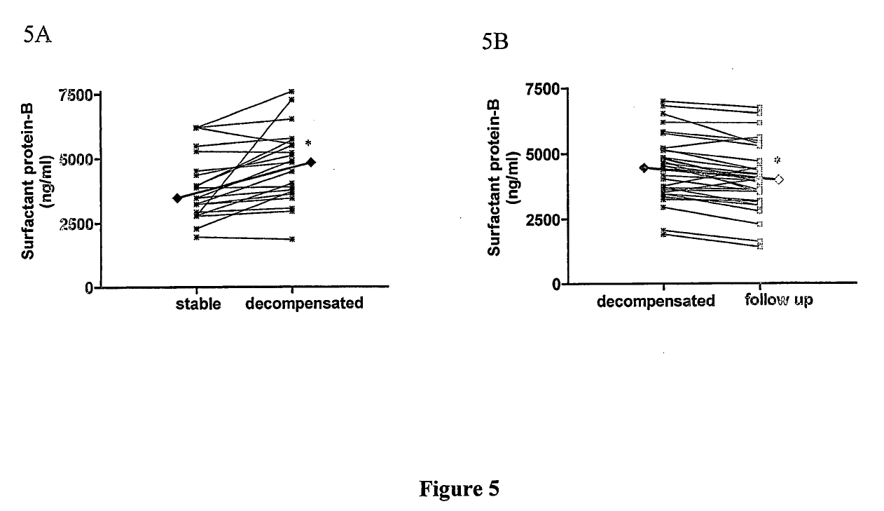

On 21 occasions the treating cardiologist elected to increase the CHF patient's loop-diuretic dosage. Comparison of measured parameters at this clinic visit compared to those at the previous visit (median of 31 days previously) revealed a decrease in dyspnea score (deterioration), an increase in left ventricular failure score, a reduction in 6-MWT distance, no change in weight, (Table 4) and an increase in NT-proBNP (FIG. 4A) and SP-B levels (FIG. 5A).

Change in Measured Parameters Following Increased Loop-Diuretic Dose.

On 32 occasions a clinic visit followed an increase in loop-diuretic dosage (median of 16 days previously). Comparison of measured parameters between these two clinic visits revealed an increase in dyspnea score (improvement), a reduction in left ventricular failure score, an increase in 6-MWT distance, a reduction in weight (Table 5) and a reduction in NT-proBNP (FIG. 4B) and SP-B levels (FIG. 51B).

### EXAMPLE 3

### Do Acute Changes in Pulmonary Microvascular Pressure, Due to Physical Exercise, Affect the Alveolocappillary Barrier?

Materials and Methods

Subjects and Procedures

Twenty consecutive subjects referred to the Veterans Heart Clinic (RGH) for exercise stress echocardiography were enrolled in the study (6 male, 14 female (mean±SEM) age 58±3 years). All subjects were referred for exclusion of exercise induced myocardial ischemia on the basis of a history of chest pain. Subjects were excluded from the study if they had a history of primary lung disease.

Subjects underwent left ventricular echocardiographic examination, had a baseline electrocardiogram performed, and had venous blood sampled for ANP and SP-B assay. In addition Doppler examination of the right ventricular outflow tract was performed. This allowed documentation of pulmonary artery flow acceleration time (pafAT) and right ventricular outflow tract velocity time integral (rVTI) as indirect indices of pulmonary hemodynamics. Pulmonary artery pressure can be derived from pafAT (pulmonary artery resistance to flow (Feigenbaum 94, Anderson 00)) and cardiac output can be derived from rVTI (directly proportional to stroke volume, and therefore for a given heart rate indicates cardiac output (Haites 84)).

The exercise stress test was then performed aiming for maximal workload (heart rate 220-age beats/min). The stress test followed the Bruce treadmill protocol (maximal test) with regular blood pressure and ECG monitoring (Gibbons 99). Immediately on completion of maximal tolerated exercise, echocardiographic examination of the left ventricle was repeated for evidence of exercise-induced left ventricular dysfunction. Repeated pulmonary outflow tract Doppler examination and venous blood sampling followed this.

Pre and immediate post (impost) echocardiographic images were assessed by a cardiologist experienced in the interpretation of echocardiographic images. Wall motion at rest and at impost was scored on a 1 to 5 scale (1=normal, 2=hypokinetic, 3=akinetic, 4=dyskinetic, and 5=aneurysm) according to the 16-segment model of the American Society of Echocardiography (Schiller 89). Wall motion score index (WMSI) was determined at rest and at peak exercise as the sum of the segmental scores divided by the number of visualized segments.

Standard ECG criteria for myocardial ischemia were utilized. The exercise ECG was considered to show evidence of ischemia if there was horizontal or downsloping ST segment depression≧1 mm at 60 msec after the J point or ≧1 mm elevation of the J point with a horizontal or upsloping ST segment lasting 60 msec.

The third criteria for exercise-induced myocardial ischemia was complaint of chest pain or excessive dyspnea with exertion. The exercise stress test was classified as positive or negative for exercise induced myocardial ischemia by the treating cardiologist on the basis of these three factors (echocardiographic exercise induced ventricular dysfunction, ECG criteria of ischemia and subjective symptoms with exercise).

Specimen Handling and Assay

Venous blood was collected in lithium heparin and ethylenediaminetetraacetate dihydrate (EDTA) tubes, centrifuged at 5000 rpm for 5 minutes at 15° C. and the supernatant frozen at −70° C. for subsequent blinded SP-B and ANP (Oliver 88) batch analysis respectively.

Statistical Analysis

As neither circulating SP-B nor ANP were normally distributed (Shapiro-Wilk test of normality) values were compared using the Wilcoxon signed rank test. Dichotomous variables were compared using Fisher's exact test. Statistical significance was defined as p<0.05, and results are presented as median (25^{th} and 75^{th} percentile). Correlation between variables was determined using Spearman's correlation.

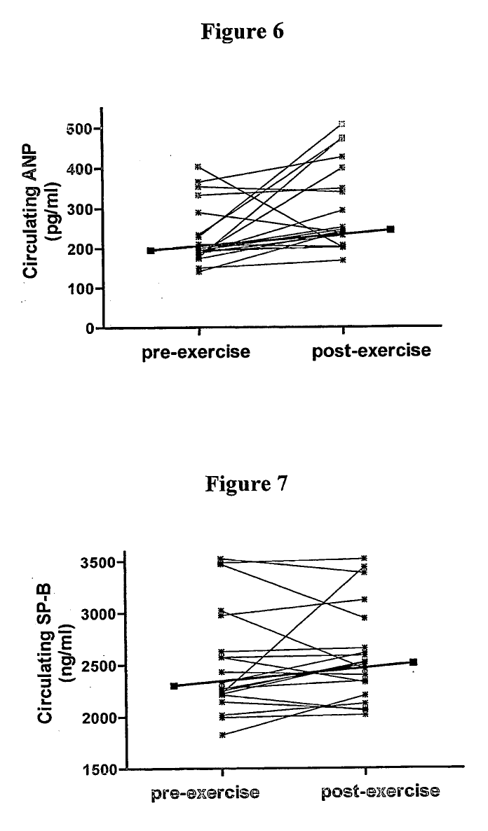

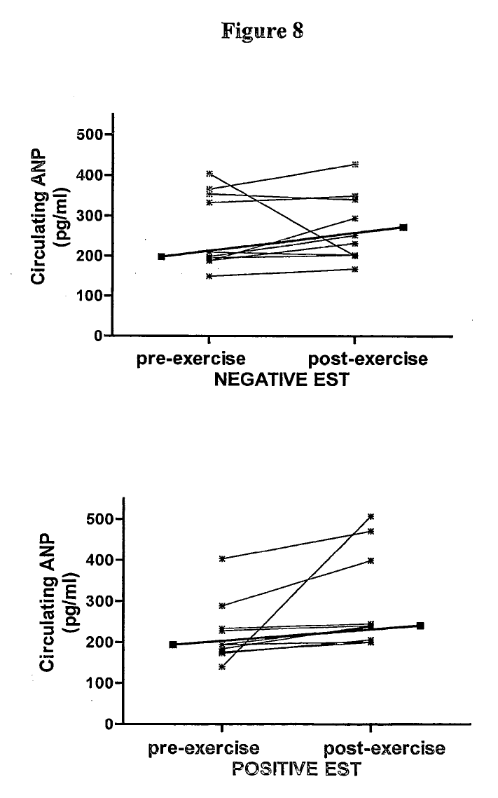

Results

Whereas circulating ANP increased from 195 pg/ml (185, 311) to 243 (204, 374) (FIG. 6) following exercise, there was no significant change in circulating SP-B, 2302 ng/ml (2215, 2803) to 2510 (2268, 2802) (FIG. 7).

Effect of Exercise-Induced Myocardial Dysfunction

Subjects were separated into two groups according to the presence of myocardial ischemia on EST. Ten subjects had “positive” tests for myocardial ischemia as determined by the attending cardiologist (Table 6).

a) Atrial Natriuietic Peptide

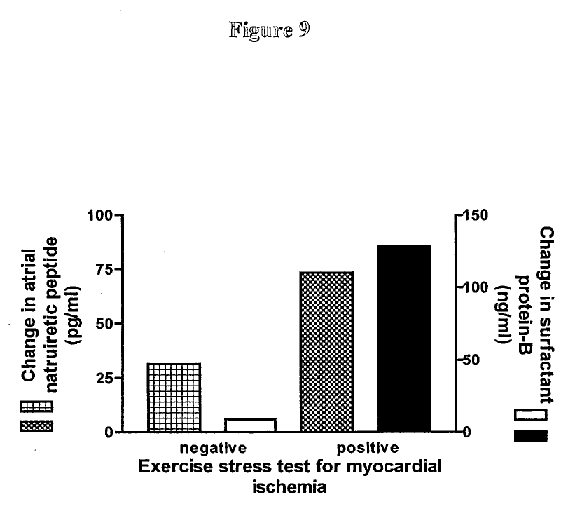

Circulating ANP was increased post-exercise in both the negative and positive EST groups (FIG. 8), although the greatest change was observed in the group with a positive EST for myocardial ischemia (FIG. 9).

b) Surfactant Protein-B

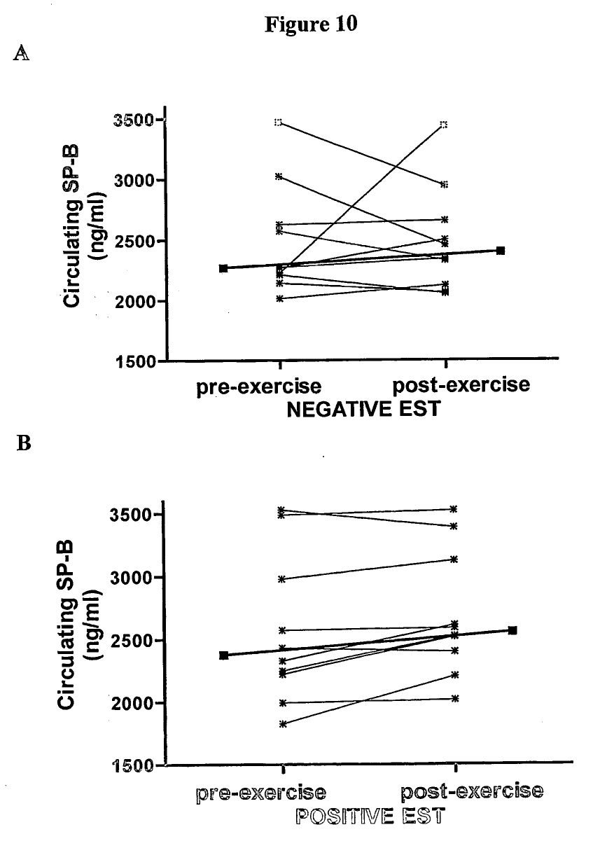

Circulating SP-B did not change post-exercise in the negative EST group (FIG. 10), however, there was a small increase post-exercise in the cohort with a positive EST for myocardial ischemia (FIGS. 9,10).

Echocardiographic Parameters of Pulmonary Vascular Pressure and Right Heart Function

Pulmonary artery flow acceleration time (pafAT) trended towards a reduction in the negative EST group following exercise, however, in the positive EST group, exercise was associated with a fall in pafAT. Although pulmonary outflow tract velocity time integral (rVTI) increased significantly in the negative EST group post-exercise, it did not change in the positive EST group (Table 7).

Correlations

There was trend towards a positive relationship between the change in ANP (ΔANP) and ΔSP-B following exercise, rₛ=0.412, p=0.07. Although there was no relationship between the ΔrVTI and ΔANP or ASP-B with exercise, there was a trend towards a negative relationship between the ΔpafAT and ΔSP-B, rₛ=−0.412, p=0.09).

### EXAMPLE 4

### Alveolocapillary Barrier Damage in Response to Acute Heart Failure: an Animal Interventional Study of Graded Acute Ascending Aorta Constriction

Materials and Methods

Placement of Ascending Aorta Snare

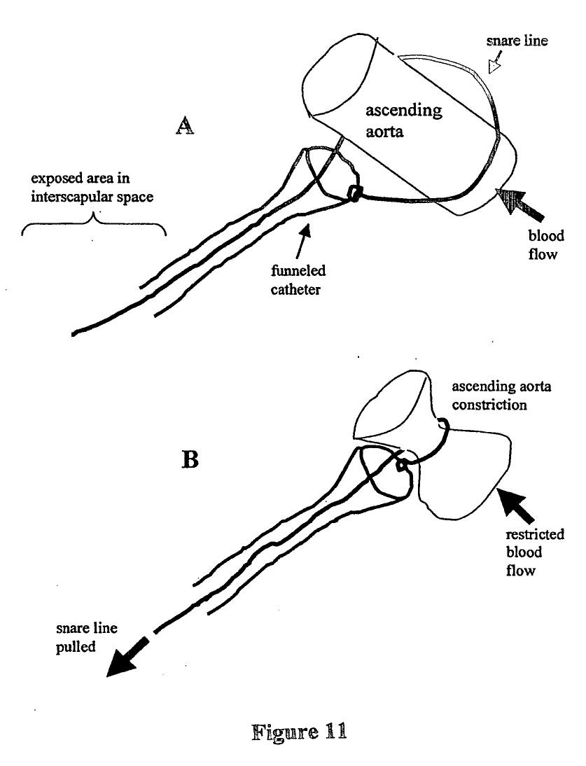

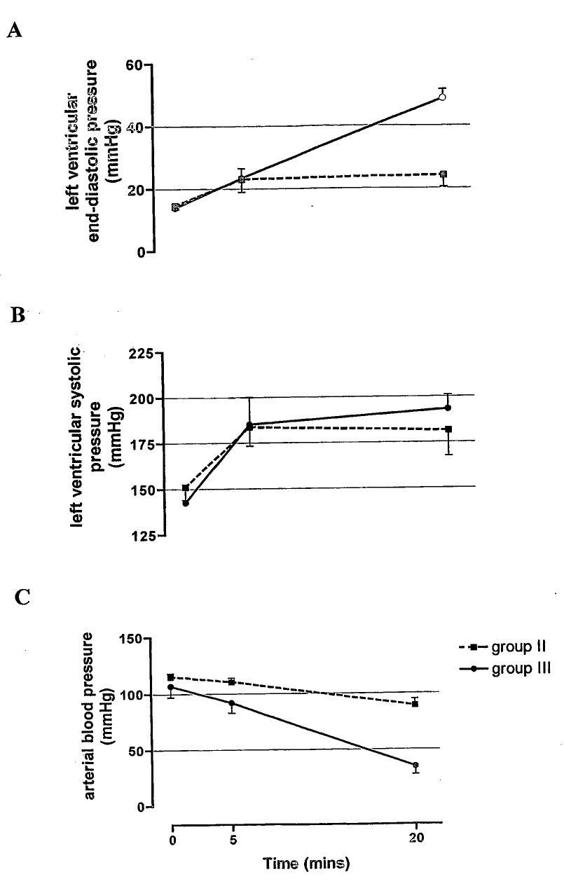

Male Sprague-Dawley rats (250-300 g) had a snare placed around the ascending aorta one-week prior to the experiment. Rats were anesthetised in an anesthetic chamber with inhaled isoflurane (3-4%, Forthrane, Abbott Australasia, Kurnell, Australia), then intubated with a 16-gauge cannula, and ventilated at 60 breaths per minute (Harvard rodent ventilator, model 683, Holliston, Mass.). Anesthesia was maintained with inhaled enflurane (1-2%, Ethrane, Abbott Australasia, Kurnell, Australia) administered through a vaporizer connected to the ventilator. Rats were placed supine on a thermostatically controlled plate to maintain body temperature at 37° C. The chest wall was shaved and a left para-sternotomy was performed through three costal cartilages. The great vessels above the heart were exposed by blunt dissection. A 3.0 prolene suture with its end tied to a funneled polyethylene catheter (OD 1 mm, ID 0.5 mm, Adelab Scientific, Norwood, Australia) was placed around the ascending aorta. The free end of the suture was threaded through the lumen of the polyethylene catheter producing a snare around the ascending aorta (FIG. 11). The free end of the polyethylene catheter with the prolene suture passing through it was tunneled subcutaneously to emerge in the interscapular space. The thorax was closed and the rats were allowed to recover from anesthesia and extubated. Intra-peritoneal buprenorphine (0.02 mg/kg, Temgesic, Reckitt and Colman, West Ryde, Australia) was administered twice daily for up to 4 days post-operatively. Control animals had the same procedure performed.

Cardiorespiratory Variables

After 1 week the rats were anesthetised with intra-peritoneal thiopentone (60 mg/kg, Pentothal, Abbott Australasia, Kurnell, Australia). The caudal artery and one lateral caudal vein at the base of the tail were cannulated with a polyethylene catheter (OD 1 mm, ID 0.5 mm)(Davidson 99) and fixed in place with tissue glue (Loctite 406, Carringbah, Australia). Anesthesia was maintained by arterial infusion of pentobarbitone (21 ml/kg/hr, Nembutal, Rhone Merieux, Pilkenba, Australia) in heparinised saline (2 U/ml; 2 ml/hr), and body temperature was maintained using a thermostatically controlled heat plate. The neck was shaved and a 15 mm vertical incision was made on the right side, 5 mm from the midline. The right carotid artery was mobilized by blunt dissection and intubated with a polyethylene catheter (OD 1 mm, ID 0.5 mm). LVEDP was monitored by advancing the catheter across the aortic valve into the left ventricle (LV).

Cardiorespiratory variables were monitored using a MacLab system 4 analog-digital instrument and Chart version 3.4.2 software (AD instruments, Sydney, Australia).

Systemic arterial blood pressure, heart rate and LV pressures were monitored with disposable pressure transducers (Sorensen Trans Pac, Abbott Critical Care Systems, Chicago, Ill.). Arterial blood gases were analyzed with an ABL 5 blood gas analyzer (Radiometer, Copenhagen, Denmark).

Tightening of Aortic Snare

The aortic snare line (prolene suture) was pulled through the polyethylene catheter at the back of the neck, tightening the snare around the ascending aorta (FIG. 11). Three experimental groups were formed; group I, (controls) where the snare line was not tightened, group II where the snare was moderately tightened, so as to increase left ventricular systolic pressure (LVSP) by 20-30%, and group III where the snare was severely tightened, so as increase baseline left ventricular systolic pressure (LVSP) by >40%. Cardiorespiratory variables were monitored for 20 minutes prior to the final data collection.

Experimental Procedure

Time 0 (T0) was defined as the time when all lines were in situ and the experiment was commenced. At T0 0.7 ml of blood was withdrawn from the arterial line, 0.1 ml was used for arterial blood gas analysis (ABG) and 0.6 ml was retained for SP-B determination. The volume was replaced with 0.7 ml of saline to maintain euvolaemia. Twelve rats then had the their snares tightened so as to increase LVSP by 20-30%, 7 rats did not have their snares tightened, group I (controls).

Five minutes after tightening the snare (T5) hemodynamics were recorded, 0.7 ml of blood was again removed for ABG and SP-B determination, with the volume again replaced with saline. Six of the 12 rats then had their snares tightened further to increase the LVSP to >40% of the original baseline, group III, (APE). The remaining six rats with no further snare tightening were designated group II.

At T10 all rats had radiolabelled permeability markers infused, and after 10 minutes, at T20, sample and cardiorespiratory variables were collected (Table 8).

Triple Radiolabel Study

The triple radiolabel technique for the study of alveolocapillary barrier permeability has been previously reported (Davidson 00).

a) Preparation of radiolabeled red blood cells. Approximately 1.2 ml of blood was drawn from male Sprague Dawley donor rats into a syringe containing heparin (5000 U/ml, 75 μl) and acid citrate-dextrose (12.25 g glucose, 11 g sodium citrate, and 4 g citric acid/500 ml, 140 μl), and was centrifuged at 6000 rpm for 10 minutes. The plasma was discarded, and the cells re-suspended in 40 μl of phosphate-buffered saline (PBS) and 4 g of acid-citrate dextrose. Sodium chromate (⁵¹Cr, 15 μCi per 100 g of recipient rat) was added, and the cells incubated at room temperature for 1 hour. The labeled cells (⁵¹Cr-red blood cells (RBCs)) were pelleted as described above, washed three times in 0.7 ml of PBS, and resuspended in PBS to 1 ml.

b) Preparation and infusion of radiolabeled albumin and diethylenetriamine pentaacetic acid. Human serum albumin labeled with ¹²⁵I (¹²⁵I-albumin; ICN Biomedicals Australasia, Sydney, Australia) and ^{99m}Tc labeled diethylenetriamine pentaacetic acid (^{99m}Tc-DTPA) (gift from the Department of Nuclear Medicine, Flinders Medical Centre, Adelaide, Australia), (1 and 20 μCi, respectively, per 100 g of recipient rat) were added to the ⁵¹Cr-RBC (1.2 μL/g body weight) and infused over 10 seconds via the caudal vein, 10 minutes before the heart and lungs were isolated.

c) Compartmentalization of radiolabels. The trachea was cannulated (16 guage cathetre) immediately following T20, and the lungs were ventilated with air at 60 cycles/min with a tidal volume of 7 ml/kg (Flexivent small animal ventilator, SCIREQ, Montreal, Canada). The thorax was rapidly opened through a para-sternotomy and 4-5 ml whole blood sampled from the LV. Plasma was separated by centrifuging at 6000 rpm for 10 minutes. The lungs and heart were removed from the thorax, a procedure that took <2 minutes, and the right upper lobe was tied off with a 3.0 prolene suture and resected. The remaining lung was degassed at 0.5 atm for 60 seconds and lavaged at 2° C. with three separate 32 ml/kg-volume aliquots of cold saline, with each volume instilled and withdrawn three times.

Radiolabels were counted in whole blood, plasma, lavage and right upper lobe with a Cobra 5003 gamma counter (¹²⁵I, 15-75 keV; ^{99m}Tc, 90-190 keV; ⁵¹Cr, 240-400 keV; Auto-gamma 5000 series, Packard Instruments, Downers Grove, Ill.). Because ^{99m}Tc interferes with the counting of ¹²⁵I and ⁵¹Cr, the later two labels were recounted ≈3 days later, after the ^{99m}Tc had decayed (half-life ≈6 hours).

Compartmentalization of the radiolabels in the whole lung and lavage was expressed as percent volume.

$$\% erythrocyte volume = \frac{[ lavage aliquot or section cpm ( {}^{51} Cr ) \text{/} ml or g wet tissue ]}{[ blood cpm ( {}^{51} Cr ) \text{/} ml ]} ⨯ 100$$

$$\% albumin volume = \frac{[ lavage aliquot or section cpm ( {}^{125} I ) \text{/} ml or g wet tissue ]}{[ plasma cpm ( {}^{125} I ) \text{/} ml ]} ⨯ 100$$

$$\% DTPA volume = \frac{[ lavage aliquot or section cpm ( {}^{99 m} Tc ) \text{/} ml or g wet tissue ]}{[ plasma cpm ( {}^{99 m} Tc ) \text{/} ml ]} ⨯ 100$$

SP-B Assay in Plasma and Lavage

In order to free the surfactant protein-B (SP-B) from any associated plasma or surfactant components, aliquots were first treated with EDTA, SDS, and Triton X-100 (Doyle 97). SP-B was determined using a human based ELISA inhibition assay (Doyle 97). The antibody reacts strongly with rat SP-B (Yogalingam 96). All samples were assayed in duplicate at 4 serial dilutions. Standards, assayed in quadruplicate, were included in each ELISA plate at 8 serial dilutions (ranging from 7.8 to 1000 ng/ml, r>0.99)

Correction for Dilution

As can be seen from Table 11 a significant percentage of rat intravascular volume was removed at each sampling time. To avoid any effects of hypovalaemia this volume was replaced with normal saline. However, repeated sampling and dilution with saline, particularly in groups II and III, would inevitably reduce the true circulating solute concentration (SP-B). This was corrected as follows, for each dilution episode:

(i) rat total blood volume was calculated (6 ml/100 g rat body weight (Waynforth 80))

(ii) remaining blood volume after sampling=(i)−0.7 ml

(iii) remaining plasma after sampling=(ii)*0.53 ml (hematocrit 47% (Waynforth 80))

(iv) dilutional increase in plasma volume=((iii)+0.7)/(iii)

(v) corrected SP-B concentration=measured concentration*(iv)

Statistical Analysis

All data are presented as mean±SEM. SP-B levels were normally distributed (Shapiro-Wilk test of normality), hence levels were compared between groups using one-way ANOVA, within group comparisons were made initially using repeated measures ANOVA, and then pairwise t tests against group I. Other measured parameters were not normally distributed hence between group comparisons were made using the Kruskal-Wallis test, within group comparisons using the Friedman test, and pairwise comparisons using the Mann Whitney U test against group I. Statistical significance was defined as p<0.05.

Results

Hemodynamic Pressure Changes with Snare Manipulation

Baseline hemodynamics were similar between the groups FIG. 12). Groups II and III had similar hemodynamic pressure changes following moderate aortic constriction at T5, with a 64% increase in LVEDP. Further aortic constriction in group III resulted in a further 109% increase in LVEDP, as well as a marked reduction in mean systemic blood pressure.

Lung Composition

There was no change in wet or dry lung weights in group II (Table 9). Group III had a 138% increase in wet lung weight, and a 38% increase in dry lung weight, so that the wet-to-dry lung weight ratio increased 72%.

Arterial Blood Gas Changes

There was no difference in PaO₂ and PaCO₂ between the three groups at baseline (FIG. 13). There was a reduction in PaO₂ in group II, and to a greater degree in group III, with time, and an increase in PaCO₂ in group II.

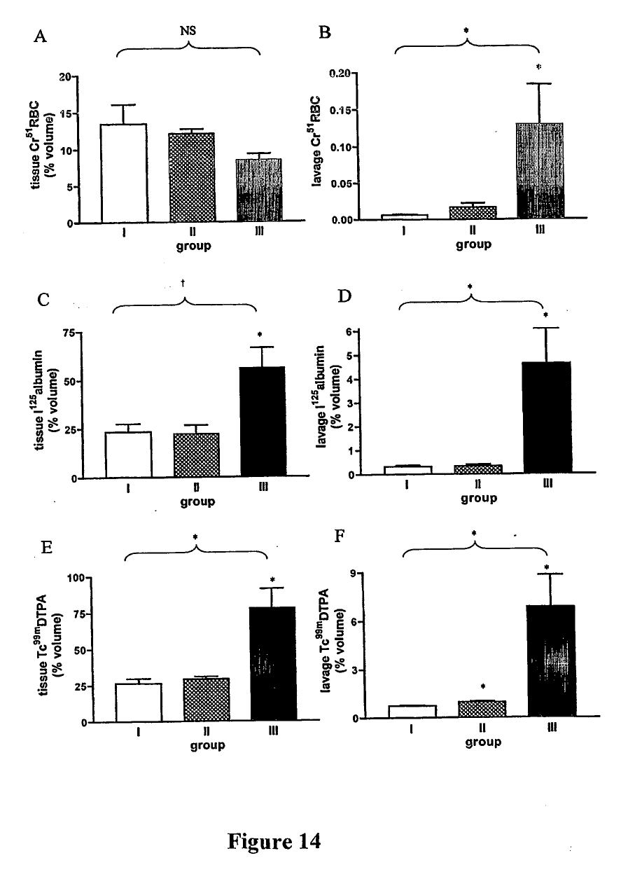

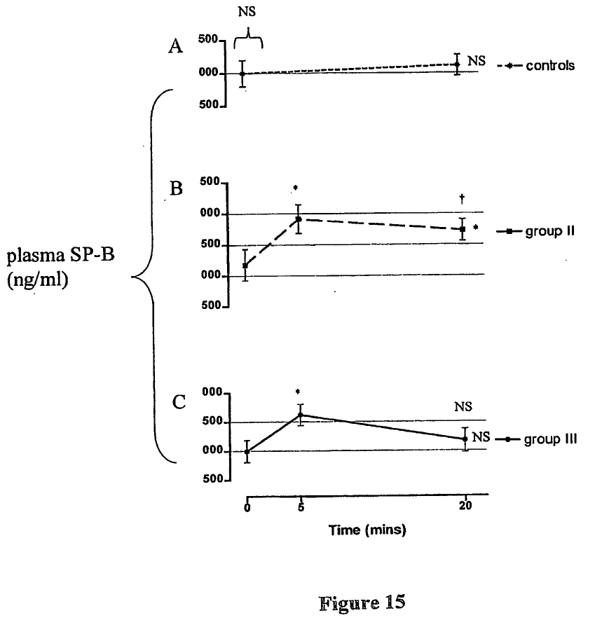

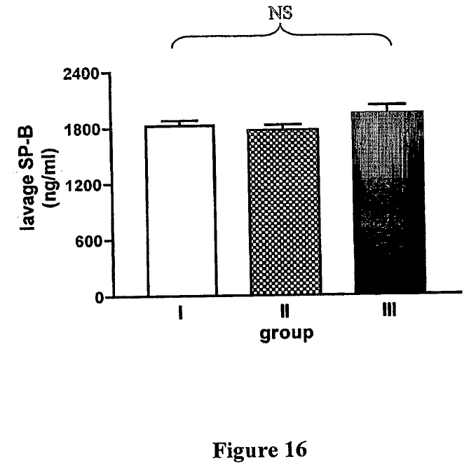

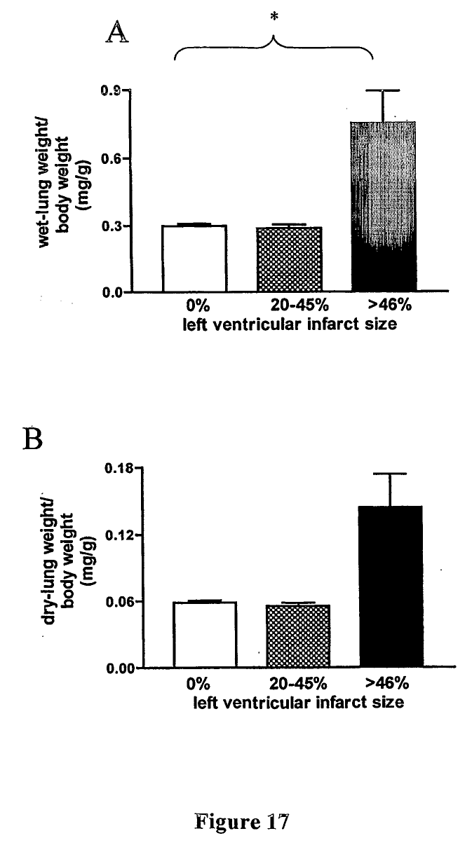

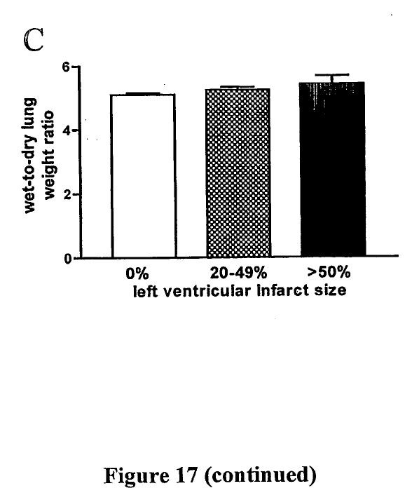

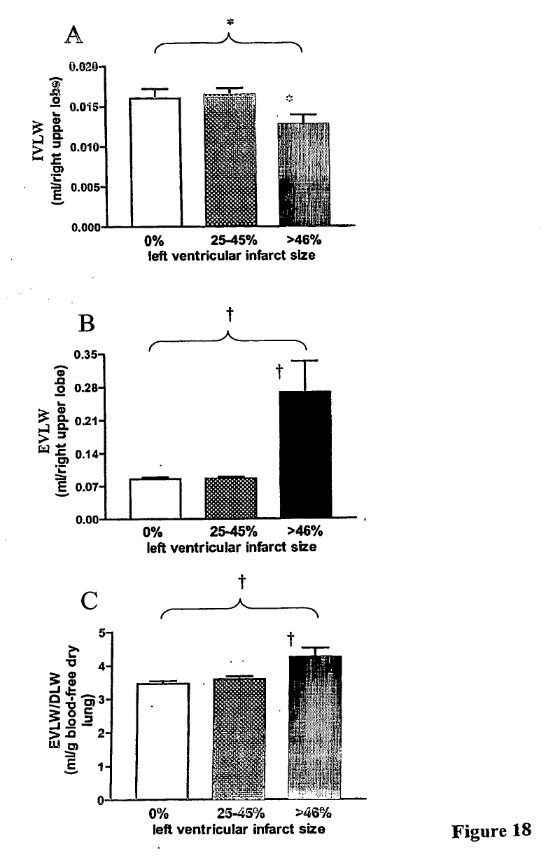

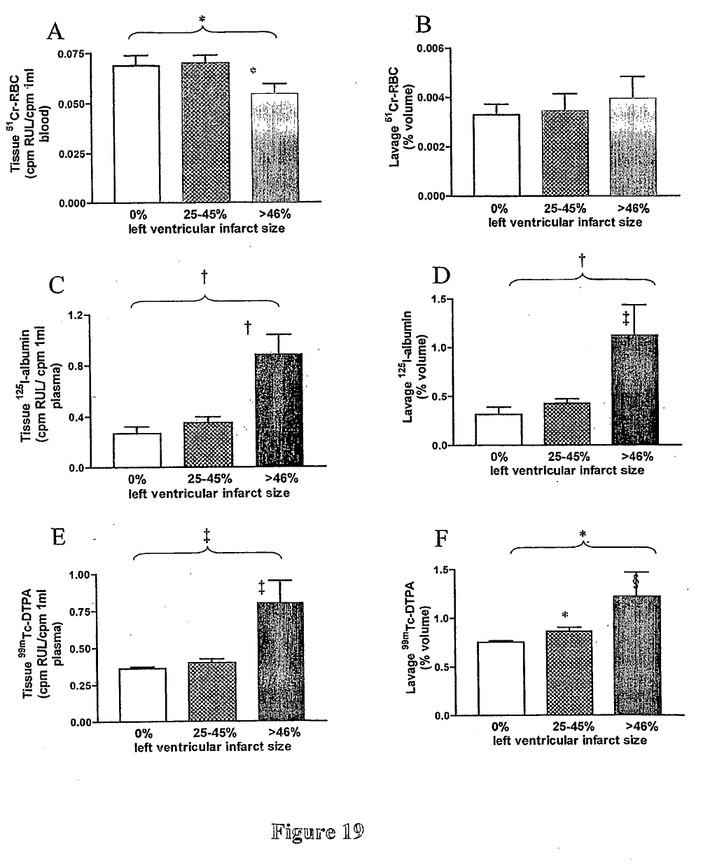

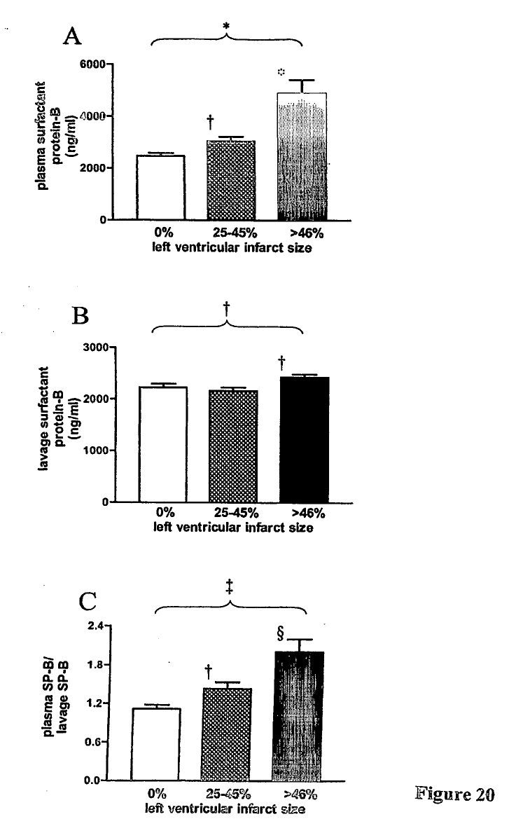

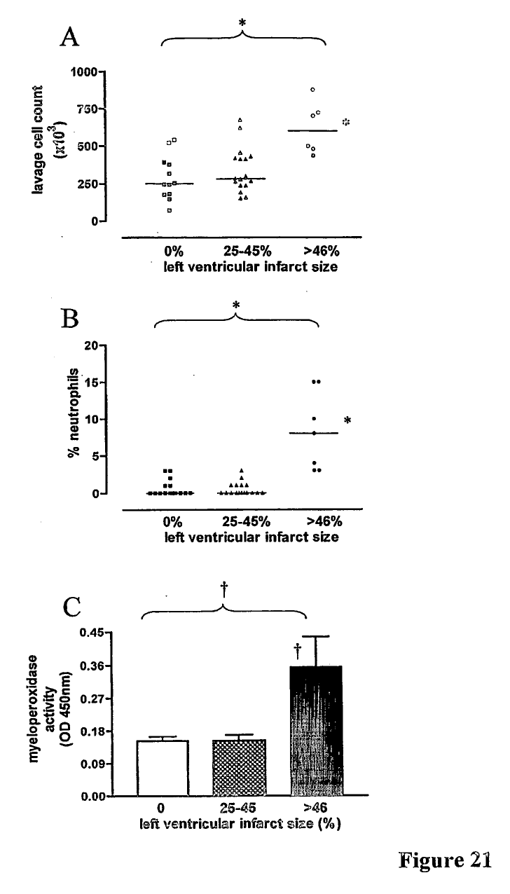

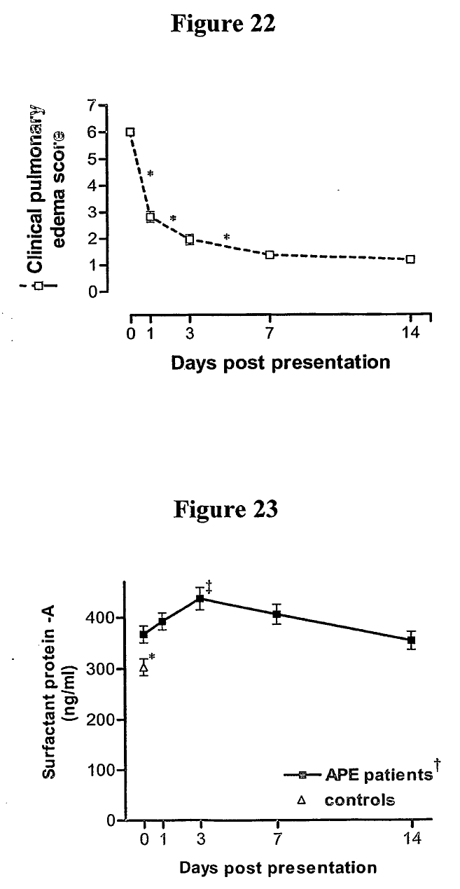

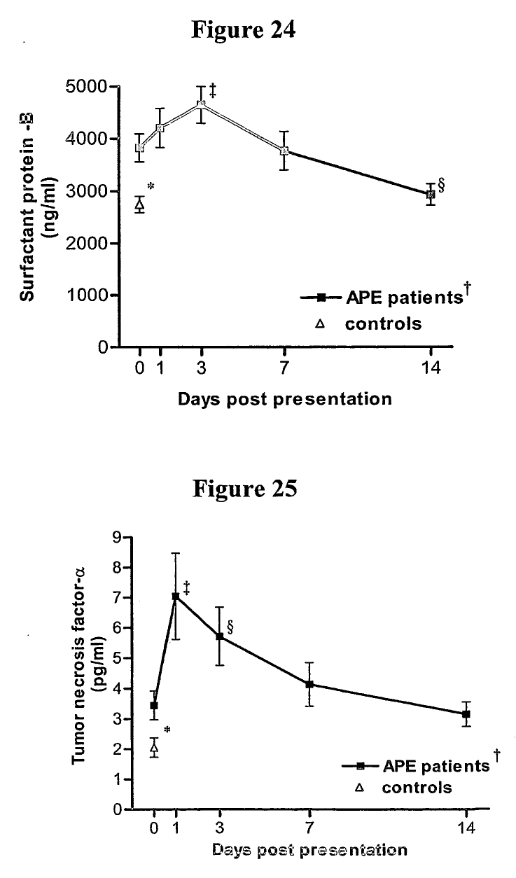

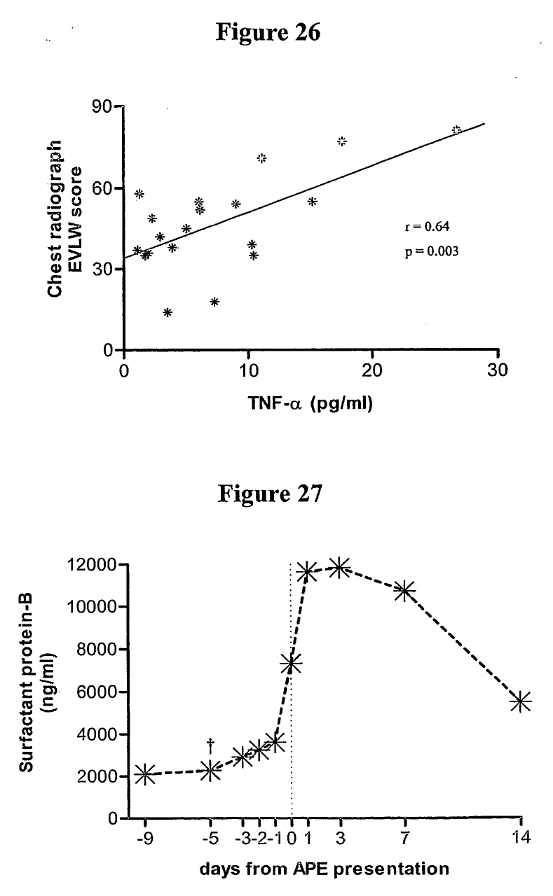

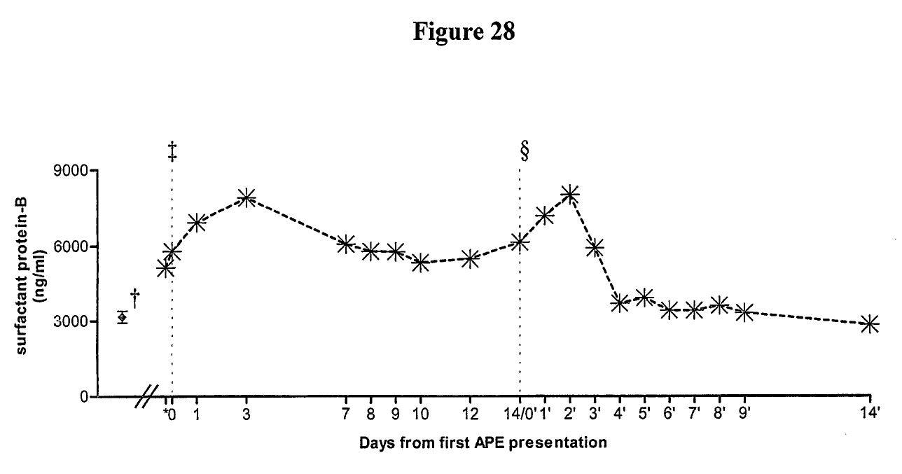

Distribution of Radiolabels (FIG. 14)

⁵¹Cr-RBC. Tissue ⁵¹Cr-RBC % volume was unchanged, but trended towards a reduction in group III. Lavage ⁵¹Cr-RBC % volume was unchanged in group II, but increased markedly in group II.

¹²⁵I-albumin. Tissue ¹²⁵I-albumin % volume was unchanged in group II, but increased 100% in group III. Lavage ¹²⁵I-albumin % volume was unchanged in group II, but increased markedly in group III.

^{99m}Tc-DTPA. Tissue ^{99m}Tc-DTPA % volume was unchanged in group II, but increased 196% in group III. Lavage ^{99m}Tc-DTPA % volume was also unchanged in group II, but increased markedly in group III.

SP-B Changes with Snare Manipulation

Circulating SP-B levels were similar in the three groups at baseline (FIG. 15). At T5 following moderate aortic constriction in groups II and III there was a 21% increase in plasma SP-B levels which was maintained at T20 in group II. In group III following further aortic constriction there was a fall in plasma SP-B so that SP-B was no longer significantly elevated over baseline levels. Lavage SP-B levels were similar between the three groups (FIG. 16).

### EXAMPLE 5

### Alveolocapillary Barrier Damage in Chronic Heart Failure; an Animal Interventional Study in an Infarct Model of Chronic Heart Failure

Materials and Methods

Induction of Heart Failure

Left ventricular myocardial infarction was induced in male Sprague-Dawley rats (250-300 g) by a modification of the method of Pfeffer and co-workers (Pfeffer 79). Rats were anesthetized in an anesthetic chamber with inhaled isoflurane (3-4%, Forthrane, Abbott Australasia, Kurnell, Australia), then intubated with a 16-gauge cannula, and ventilated at 60 breaths per minute (Harvard rodent ventilator, model 683, Holliston, Mass.). Anesthesia was maintained with inhaled enflurane (1-2%, Ethrane, Abbott Australasia, Kurnell, Australia) administered through a vaporizer connected to the ventilator. Rats were placed supine on a thermostatically controlled plate to maintain body temperature. The chest wall was shaved and a left para-sternotomy was performed through three costal cartilages. The pericardium was opened and the left coronary artery ligated between the pulmonary artery outflow tract and the left atrium with a 6.0 prolene suture. The thorax was closed and the rats were allowed to recover from anesthesia and extubated. Intra-peritoneal buprenorphine (0.02 mg/kg, Temgesic, Reckitt and Colman, West Ryde, Australia) was administered twice daily for up to 4 days post-operatively.

Cardiorespiratory Variables

After 7 weeks the rats were prepared for cardiorespiratory monitoring as described in Example 3.

Triple Radiolabel Study

a) Preparation of Radiolabeled Red Blood Cells, b) Preparation and Infusion of Radiolabeled Albumin and Diethylenetriamine Pentaacetic Acid, and c) Compartmentalization of Radiolabels were Performed as Described in Example 3.

As CHF is a chronic condition with pulmonary remodeling in response to chronic exposure to high Pₘᵥ (Lee 79), the compartmentalization of the radiolabels in whole lung tissue (right upper lobe) was expressed differently to Example 3 to allow for the differences in lung parenchymal composition, hence;

$$Tissue erythrocytes = \frac{[ right upper lobe counts per minute ( cpm ) ( {}^{51} Cr ) ]}{[ blood cpm ( {}^{51} Cr ) \text{/} ml ]}$$

$$Tissue albumin = \frac{[ right upper lobe cpm ( {}^{125} I ) ]}{[ plasma cpm ( {}^{125} I ) \text{/} ml ]}$$

$$Tissue DTPA = \frac{[ right upper lobe cpm ( {}^{99 m} Tc ) ]}{[ plasma cpm ( {}^{99 m} Tc ) \text{/} ml ]}$$

Compartmentalization of the radiolabels in the lavage was expressed as percent volume as described in Example 3.

Quantification of Heart Failure

At the termination of the experiment the right and left ventricles (RV and LV) were dissected, separated and weighed (the RV was dissected off the septum). The LV was preserved in formaldehyde before being cut into four transverse sections for planimetric determination of total circumferential infarct size as a percentage of total LV circumference (Pfeffer 79). As the left coronary artery cannot be visualized directly during surgery, there was no myocardial infarction in some animals. Infarcts were therefore graded as nil (0% LV infarction, controls), moderate (25-45%) and large (>46%).

Lavage Fluid Total Protein

Total protein concentration in the lavage fluid was determined by a modification of the Lowry method (Doyle 94).

Markers of Airspace Inflammation

a) Cell count The lavage fluid was centrifuged at 2° C. for 5 minutes at 800 rpm. A cell count was performed on the pellet using a hemacytometer (Improved Neubauer BS 748, Weber Scientific International, Teddington, UK), and a differential cell count was performed after staining with Diff-Quik and Papanicolaou stains.

b) Myeolperoxidase activity. Bronchoalveolar lavage fluid myeloperoxidase activity was quantified using the method of Schneider and Issekutz (Schneider 96). The optical density at 450 nm was measured at five minutes using a Dynatech plate reader (Dynatech laboratories, Chantilly, Va.)

SP-B Assay in Plasma and Lavage

Plasma and lavage fluid SP-B was determined using a human based ELISA inhibition assay.

Measurement of Intra and Extravascular Lung Water (IVLW, EVLW)

The right upper lobe and an aliquot of whole blood were counted for ⁵¹Cr-RBC, both were frozen, dried (−50° C., Maxi-dry, FTS Systems, Stone Ridge, N.Y.) and weighed. Intravascular lung water per right upper lobe (IVLW), EVLW and blood-free dry lung weight (DLW) were calculated using a modification of the method described by Pearce and co-workers (Pearce 65, Kirk 69) to allow expression of EVLW/DLW as milliliters per gram blood-free dry weight.

Statistical Analysis

Data is presented as mean±SEM unless otherwise indicated. Normally distributed data (Kolmogorov-Smirnov test of normality) was analyzed using one-way ANOVA, and post hoc pairwise t tests against the control group with modified Bonferroni correction (Wallenstein 80). Remaining data was analyzed using the Kruskal-Wallis test, and post hoc comparisons with the Mann-Whitney U test with modified Bonferroni correction (Wallenstein 80). Statistical significance was defined as p<0.05.

Results

Of the 39 rats studied, LV histology revealed 15 rats with no myocardial infarction (control group). Seven rats had large infarcts (55±4% LV circumference) and 17 had moderate infarcts (35±2% LV circumference). Rat weights were similar at the terminal experiment (controls; 427±1 g, moderate infarct; 448±1 g, large infarct; 435±3 g), p=0.64.

Physiologic Evidence of Congestive Heart Failure

Heart rate and mean arterial blood pressure were similar between the three groups, however, LVEDP increased progressively with infarct size (p<0.001) (Table 10). Although there was no difference in right ventricular (RV) weight between the controls and the moderate infarct group, RV weight increased 73% in the large infarct group (p<0.001). In contrast LV weight was unchanged over the three groups.

Although the partial pressure of oxygen in arterial blood (PaO₂) fell as infarct size increased (p<0.01) (Table 10), there was no difference in the pH or partial pressure of carbon dioxide in arterial blood between the groups.

Lung Composition

While the wet-lung weight was not changed in the moderate infarct group, it was elevated 152% in the large infarct group (p<0.001) (FIG. 17A), and this increase was matched by an increase in dry-lung weight (p<0.001) (FIG. 17B), such that tie wet-to-dry lung weight ratio was unchanged across groups (FIG. 17C). Derived values of lung water (Pearce 65, Kirk 69) were consistent with these weight changes in the large infarct group, with a 215% increase in EVLW (p<0.001) (FIG. 18B), partially offset by a 20% reduction of IVLW (p<0.05) (FIG. 18A). EVLW/DLW increased 23% in the large infarct group (p<0.001) (FIG. 18C).

Tissue and Lavage Radiolabel Compartmentalization

⁵¹Cr-RBC. Tissue ⁵¹Cr-RBC was reduced 20% in the large infarct group (p<0.05) (FIG. 19A). The percentage of ⁵¹Cr-RBC per ml of lavage fluid remained constant, and at very low levels, over the three groups (FIG. 19B).

¹²⁵I-albumin. There was a 230% increase in tissue ¹²⁵I-albumin in the large infarct group (p<0.001) (FIG. 19C). The percentage of ¹²⁵I-albumin per ml of lavage fluid also increased markedly (250%) in this group (p<0.01) (FIG. 19D).

^{99m}Tc-DTPA. There was a 120% increase in tissue ^{99m}Tc-DTPA in the large infarct group (p<0.01) (FIG. 3E). However, the percentage of ^{99m}Tc-DTPA per ml of lavage fluid increased only 60% in this group (p<0.025) (FIG. 19F).

Total Protein in Lavage Fluid

Lavage fluid total protein was similar in the control (0.14±0.01 mg/ml) and moderate infarct groups (0.15±0.02). However, there was a 350% increase in the large infarct group (0.644±0.17) (p<0.001).

Surfactant Protein-B

Plasma SP-B increased progressively with infarct size (p<0.001). There was a 23% increase in the moderate infarct group (p<0.05), and a further 60% increase in the large infarct group (p<0.001) (FIG. 20A). Lavage SP-B was unchanged in the moderate infarct group but did increase slightly (9%) in the large infarct group (p<0.05) (FIG. 20B).

Lavage Cytology

Although unchanged in the moderate infarct group, the lavage cell count was increased in the large infarct group (p<0.01) (FIG. 21A), with increased numbers of neutrophils (p<0.01) (FIG. 21B). Furthermore, myeloperoxidase activity was increased in the cell-free lavage fluid from the large infarct group (p<0.05) (FIG. 21C).

### EXAMPLE 6

Materials and Methods

The Flinders Medical Centre Committee on Clinical Investigation approved this study (permit 74/99), and all subjects gave written informed consent to participation.

Subjects and Procedures.

Twenty-eight consecutive patients with APE requiring mask continuous positive airway pressure for acute respiratory failure (Bersten et al., N Engl J Med 1991; 325:1825-1830) were studied (16 women and 12 men; mean±SEM age, 75±2 yrs). All patients had a clinical diagnosis of APE, including sudden onset of dyspnea and diaphoresis with tachycardia, tachypnea, hypertension, widespread pulmonary crepitations, and acute respiratory failure, in the absence of fever. Exclusion criteria included an acute history of aspiration or infection, ST segment elevation myocardial infarction, primary lung disease, and concurrent inflammatory disease. Patients received standard treatment for APE comprising intravenous glycerol trinitrate, furosernide and morphine, and mask continuous positive airway pressure with high F_{IO}_{₂}.

Venous blood for SP-A and -B, TNF-α assay, and plasma creatinine measurement was collected on days 0 (presentation), 1, 3, 7, and 14. Patients were graded for clinical evidence of pulmonary edema at the same time points using a clinical pulmonary edema scoring system (Killip et al., Am J Cardiol 1967; 20:457-464) (Table 6). Chest radiographs were performed on presentation and on day 3. Radiographs were graded for extravascular lung water (EVLW) (Pistolesi et al., Radiol Clin North Am 1978; 16:551-574) in a blinded fashion by a radiologist. Arterial blood was collected via a radial artery for blood gas analysis (Radiometer Copenhagen ABL 620, 1998, Copenhagen, Denmark) at presentation and again within 2 hrs. Thirteen age-matched, normal volunteers, free from any cardiorespiratory or in-flammatory disease, were used as a control group (nine women and four men; age, 78±4 yrs).

Specimen Handling and Assays.

Venous blood was collected in lithium heparin tubes, centrifuged at 5000 rpm for 5 mins at 15° C., and the supernatant frozen at −70° C. SP-A and -B levels were measured using a competitive ELISA as previously described (Doyle et al., Am J Respir Crit Care Med 1995; 152:307-317; Doyle et al., Am J Respir Crit Care Med 1997; 156: 1217-1229). TNF-α levels were measured using a high sensitivity immunoassay (Biosource International, Human TNF-α US ultrasensitive, Camarillo, Calif.; range, 0.5-32 pg/mL; sensitivity, <0.09 pg/mL; precision coefficients of variation: inter-assay<7%, intra-assay<5%, recovery 103% from normal human plasma).

Statistical Analysis. Data were analyzed using SPSS for Windows release 10.0 (SPSS, Chicago, Ill.). The distribution of SP-A and -B and TNF-α levels were skewed (Kolmogorov-Smirnov); hence, data were logarithmically transformed and analyzed using parametric statistics. All data are presented as mean±SEM, with statistical significance defined as p<0.05. Differences in SP-A and -B, and TNF-α levels between controls and the APE subjects on day 0 were tested using Student's t-test. Repeated measures analysis of variance was used to test for a difference in means over the five sampling times. Contrasts with correction for repeated analysis were used to determine changes between mean levels from the first sampling point (Wallenstein et al., Circ Res 1980; 47:1-9).

Clinical markers of pulmonary edema were analyzed using nonparametric statistical analysis (Wilcoxon's signed-ranks test). Clinical pulmonary edema scores and plasma creatinine levels at adjacent sampling times were compared using Wilcoxon's signed-rank test with Bonferroni correction (p<0.0125) (26). Correlations were performed using Spearman's rank order correlation test for nonnormally distributed data and Pearson's correlation test for normally distributed data.

Results

Baseline Characteristics.

All APE patients had a history of CHF, 61% of which was secondary to ischemic heart disease. Mean premorbid echocardiographic ejection fraction was <30%. There were no Q-wave myocardial infarcts associated with APE episodes; however, 57% of patients had a rise in plasma creatine kinase MB isoform (CK-MB; reference range, <7 μg/L). Although biochemical myocardial damage was generally minor, in two patients CK-MB rose above 100 μg/L (Table 7).

Clinical, Biochemical, and Radiologic Markers of Pulmonary Edema.

Typical of APE, patients were hypertensive, tachycardic, and had markedly impaired oxygenation at presentation, and these variables improved within 2 hrs of treatment, p<0.001 (Table 8). All patients scored the maximum 6 points on the clinical pulmonary edema score on day 0 and this rapidly improved so that by day 3, the mean score was <2 (minimum score, 1), p<0001 (FIG. 22).

Chest radiograph EVLW score fell 83% from day 0 to day 3, p<0001 (Table 8). Whereas mean day 0 scores represent approximately 100 mL of EVLW per liter of total lung capacity, day 3 scores were consistent with physiologic EVLW (25).

Other Biochemical Variables.

Renal function, as determined by plasma creatinine improved from days 0 to 1 (1.36±0.11 to 1.13±0.11, p<0.0125) and then remained constant (reference range, <1.36 mg/dl).

Plasma SP-A and -B.

Plasma SP-A and -B levels at day 0 were elevated 22% and 39%, respectively, relative to the age-matched controls, p<0.05. Both SP-A and -B rose further, to peak at day 3, with levels approximately 44% and 69% higher than controls (p<0.001 and p<0.01, respectively). Both SP-A and -B fell thereafter; SP-B was again more dynamic, being 7% higher than controls at day 14, whereas SP-A was still elevated 17% (FIGS. 23 and 24).

SP-B levels reflected severity being related to presentation PaO₂/F_{IO}_{₂} ratio on days 0 (rₛ=0.593, p=0.002) and 3 (rs=0.518, p=0.008). Furthermore, there was a weak correlation between the clinical pulmonary edema score and SP-B during resolution on days 1 (rₛ=0.376, p=0.048) and 3 (rₛ=0.406, p=0.041).

Plasma TNF-α Levels.

Plasma TNF-α levels were elevated over controls at day 0 (p<0.05), doubled by day 1 (p<0.05), and remained elevated by 69% at day 3 (p<0.05); levels on days 0, 7, and 14 were similar (FIG. 25).

TNF-α was unrelated to age, New York Heart Association class, ejection fraction, peak CK-MB, plasma creatinine, PaO₂/F_{IO}_{₂}, or SP-A and -B. Day 1 TNF-α levels (peak) correlated with presentation chest radiograph EVLW (rₚ=0.64, p=0.003) (FIG. 5) but day 3 TNF-α levels did not (rₚ=0.23, p=0.3).

Case Studies

Case 1.

A 58-yr-old man with a history of coronary artery disease presented 9 days before the index APE episode (day −9) with unstable angina. Despite medical therapy, he had an acute myocardial infarct on day −5, with a peak CK-MB of 389 μg/L. He was clinically stable from days −3 to −1, with no clinical evidence of left ventricular failure. On day 0, he developed APE, with no preceding chest pain or arrhythmia, and CK-MB peaked at 24 μg/L. He improved with the institution of APE treatment.

Plasma SP-B remained static until the myocardial infarction, when it slowly, progressively increased until the episode of APE. At this time SP-B suddenly increased several-fold, remained markedly elevated for 7 days, and then declined by day 14 (FIG. 27).

Case 2.

A 53-yr-old man with an 8-yr history of CHF secondary to idiopathic dilated cardiomyopathy was admitted with a 1-wk history of worsening CHF (dyspnea, weight gain, and basal pulmonary crepitations). Six hours later, he developed APE. There was initial rapid clinical improvement with routine APE therapy. After treatment and a convalescent phase in the hospital, he lost 6 kg in body weight. There was no clinical evidence of left ventricular failure for several days, until day 14, when he again developed APE, without any apparent clinical precipitant. Again, he improved rapidly after more aggressive treatment (a further 4 kg weight loss) and had no further complications.

Plasma SP-B was elevated at presentation, with worsening CHF, and rose further 6 hrs later with the onset of APE, finally peaking at day 3. Of note, SP-B remained elevated at approximately presentation levels through to at least 12. After recurrence of APE, SP-B increased again before falling to baseline levels from day 4. (FIG. 28).

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

TABLE 1
Clinical parameters and event rate across NYHA classification.

| | NYHA functional classification | | | |
|---|---|---|---|---|
| | class II | | class IV | |
| | (n = 17) | class III (n = 22) | (n = 14) | p |
| age | 62 ± 3 | 66 ± 2 | 73 ± 3 | 0.017 |
| DS | 7.8 ± 0.5 | 5.4 ± 0.3 | 4.3 ± 0.6 | <0.001 |
| LVFS | 0.23 ± 0.1 | 0.77 ± 0.2 | 2.1 ± 0.4 | <0.001 |
| 6-MWT (m) | 479 ± 27 | 331 ± 22 | 137 ± 35 | <0.001 |
| CHF admission | 2 (12%) | 6 (27%) | 8 (57%) | 0.009 |
| death | 0 | 2 (9%) | 7 (50%) | <0.001 |

Abbreviations are: dyspnea score (DS), left ventricular failure score (LVFS), 6-minute walk test (6-MWT),

TABLE 2
Correlations between measured parameters in CHF patients

| | | | | LV | | | | |
|---|---|---|---|---|---|---|---|---|
| | | | dyspnea | failure | | | | |
| | | age | score | score | 6-MWT | SP-A | SP-B | ANP |
| age | rₛ | — | | | | | | |
| | p | | | | | | | |
| dyspnea | rₛ | −0.17 | — | | | | | |
| score | p | 0.2 | | | | | | |
| LV failure | rₛ | 0.25 | −0.46 | — | | | | |
| score | p | 0.08 | 0.001 | | | | | |
| 6-MWT | rₛ | −0.57 | 0.64 | −0.46 | — | | | |
| | p | 0.000 | 0.000 | 0.001 | | | | |
| SP-A | rₛ | 0.02 | −0.24 | 0.30 | −0.23 | — | | |
| | p | 0.9 | 0.08 | 0.028 | 0.11 | | | |
| SP-B | rₛ | 0.15 | −0.39 | 0.50 | −0.50 | 0.29 | — | |
| | p | 0.3 | 0.005 | 0.000 | 0.000 | 0.033 | | |
| ANP | rₛ | 0.53 | −0.15 | 0.29 | −0.52 | −0.19 | −0.32 | — |
| | p | 0.006 | 0.5 | 0.15 | 0.001 | 0.4 | 0.11 | |
| NT- | rₛ | 0.33 | −0.30 | 0.40 | −0.41 | −0.05 | 0.46 | 0.75 |
| proBNP | p | 0.07 | 0.10 | 0.024 | 0.029 | 0.8 | 0.009 | 0.001 |

Abbreviations are:
left ventricular (LV),
6-minute walk test (6-MWT),
surfactant protein-A (SP-A),
surfactant protein-B (SP-B),
atrial natriuretic peptide (ANP),
N terminal pro brain natriuretic peptide (NT-proBNP).

TABLE 3
Plasma SP-B and NT-proBNP in matched cohorts of
CHF patients split for CHF hospitalization and death

| | CHF | no CHF | | | | |
|---|---|---|---|---|---|---|
| | hospitalisation | hospitalisation | p | death | survival | p |
| SP-B | 4841 ± 258 | 3918 ± 252 | 0.002 | 5138 ± 381 | 4004 ± 218 | 0.009 |
| NT- | 13191 ± 5367 | 3441 ± 1014 | 0.023 | 22057 ± 8478 | 3367 ± 1096 | 0.006 |
| proBNP | | | | | | |

Abbreviations are:
surfactant protein-B (SP-B),
N-terminal pro brain natriuretic peptide (NT-proBNP).
Units are:
SP-B ng/ml,
NT-proBNP pg/ml

TABLE 4
Change in clinical parameters with decision to increase diuretic

| | Heart failure clinic visit | | |
|---|---|---|---|
| | previously stable | diuretic increased | p |
| NYHA class | 3 (3, 3) | 3 (3, 4) | 0.002 |
| body weight (kg) | 82 (61, 84) | 80 (64, 86) | 0.19 |
| dyspnea score | 5 (4, 7) | 3.5 (3, 4) | <0.001 |
| left ventricular | 0.5 (0, 1.3) | 1.8 (1.1, 3.9) | <0.001 |
| failure score | | | |
| 6-MWT (m) | 343 (235, 436) | 173 (80, 263) | <0.001 |

Abbreviations are: 6-minute walk test (6-MWT)

TABLE 5
Change in clinical parameters following diuretic dosage increase

| | Heart failure clinic visit | | |
|---|---|---|---|
| | diuretic increased | follow up visit | p |
| NYHA class | 3 (3, 4) | 3 (3, 3) | <0.001 |
| body weight (kg) | 75 (65, 84) | 73 (63, 82) | <0.001 |
| dyspnea score | 3.5 (3, 5) | 5.5 (4, 6) | <0.001 |
| left ventricular | 2 (1.5, 4) | 0.5 (0, 1) | <0.001 |
| failure score | | | |
| 6-MWT (m) | 178 (98, 276) | 350 (220, 418) | <0.001 |

Abbreviations are: 6-minute walk test (6-MWT)

TABLE 6
Subject characteristics split for EST result

| | Negative EST | Positive EST | |
|---|---|---|---|
| | (n = 10) | (n = 10) | p value |
| age | 55 (46, 61) | 56 (52, 73) | 0.48 |
| % maximum heart | 95 (83, 100) | 99 (95, 101) | 0.17 |
| rate achieved | | | |
| % maximum duration | 96 (80, 108) | 113 (88, 132) | 0.39 |
| achieved | | | |
| developed chest pain | 1 | 3 | 0.58 |
| developed dyspnea | 1 | 7 | 0.02 |
| pre-exercise WMSI | 1 (1, 1.2) | 1.2 (1, 1.3) | 0.32 |
| post-exercise WMSI | 1 (1, 1) | 1.4 (1.2, 1.6) | 0.003 |
| Change in WMSI | 0 (0, 0) | 2.4 (1.6, 4.1) | <0.001 |

Abbreviations are: WMSI, left ventricular wall motion score index

TABLE 7
Doppler parameters of right ventricular outflow following exercise

| | negative EST | | | positive EST | | |
|---|---|---|---|---|---|---|
| | (n = 10) | | | (n = 10) | | |
| | Pre-exercise | Post-exercise | p value | Pre-exercise | Post-exercise | p value |
| pafAT* (m/s) | 147 | 104 | 0.055 | 147 | 93 | 0.044 |
| | (102, 175) | (85, 140) | | (120, 193) | (87, 146) | |
| Derived mPAP* | 13 | 32 | | 13 | 37 | |
| rVTI (m) | 0.16 | 0.20 | 0.014 | 0.19 | 0.17 | 0.83 |
| | (0.14, 0.19) | (0.18, 0.21) | | (0.15, 0.21) | (0.15, 0.23) | |
| Derived minute | 25 | 32 | | 31 | 28 | |
| distance^{†} (m/min) | | | | | | |

Abbreviations are:
pafAT, pulmonary artery flow acceleration time;
mPAP, mean pulmonary artery pressure;
rVTI, right ventricular outflow tract velocity time intergral
Derived data use median values:
*mean pulmonary artery pressure (mmHg) = 79 − (0.45 × pafAT)(Mahan83)
^{†}minute distance = rVTI × heart rate ∝ cardiac output (Haites84)

TABLE 8
Experimental procedure

| | Time 0 minutes (T0) | T5 | T10 | T20 |
|---|---|---|---|---|
| Group I | 0.7 ml (ABG, SP-B) | | infusion of | final data |
| (n = 7, | | | radiolabels | collection |
| controls) | | | | |
| Group II | 0.7 ml (ABG, SP-B) | 0.7 ml | infusion of | final data |
| (n = 6, | snare tightened to | (ABG, | radiolabels | collection |
| moderate | ↑ LVSP by 20-30% | SP-B) | | |
| constriction) | | | | |
| Group III | 0.7 ml (ABG, SP-B) | 0.7 ml | infusion of | final data |
| (n = 6, | snare tightened to | (ABG, | radiolabels | collection |
| severe | ↑ LVSP by 20-30% | SP-B) | | |
| constriction) | | snare | | |
| | | tightened to | | |
| | | ↑ LVSP | | |
| | | >40% | | |

TABLE 9
Change in lung weight.

| | Group I | Group II | Group III | |
|---|---|---|---|---|
| | (n = 7) | (n = 6) | (n = 6) | p value |
| Body | 324 ± 23 | 300 ± 10 | 304 ± 5 | 0.46 |
| weight (g) | | | | |
| Wet-lung | 0.32 ± 0.02 | 0.36 ± 0.001 | 0.76 ± 0.06* | <0.001 |
| weight/ | | | | |
| body | | | | |
| weight | | | | |
| (mg/g) | | | | |
| Dry-lung | 0.068 ± 0.005 | 0.073 ± 0.004 | 0.094 ± 0.006^{†} | 0.009 |
| weight/ | | | | |
| body | | | | |
| weight | | | | |
| (mg/g) | | | | |
| Wet-to- | 4.7 ± 0.1 | 5.0 ± 0.2 | 8.1 ± 0.3* | <0.001 |
| dry lung | | | | |
| weight | | | | |
| ratio | | | | |

*p < 0.01 versus group I
^{†}p < 0.05 versus group I

TABLE 10
Changes in physiologic parameters with myocardial infarct size

| | | Moderate | | |
|---|---|---|---|---|
| | Control | infarct | Large infarct | |
| | (no infarct) | (25-45% LV) | (>46% LV) | p value* |
| Heart rate | 430 ± 7 | 418 ± 8 | 416 ± 18 | 0.58 |
| (beats/min) | | | | |
| Mean arterial | 112 ± 3 | 112 ± 4 | 98 ± 9 | 0.29 |
| blood pressure | | | | |
| (mmHg) | | | | |
| LVEDP | 11.2 ± 1.2 | 14.9 ± 1.2^{†} | 28.6 ± 2.5^{‡} | <0.001 |
| (mmHg) | | | | |
| RV weight | 0.59 ± 0.02 | 0.57 ± 0.01 | 1.02 ± 0.1^{‡} | <0.001 |
| (mg/g body | | | | |
| weight) | | | | |
| LV weight | 2.19 ± 0.05 | 2.16 ± 0.03 | 2.24 ± 0.07 | 0.55 |
| (mg/g body | | | | |
| weight) | | | | |
| PaO₂ (mmHg) | 85 ± 2 | 80 ± 2 | 69 ± 5^{†} | 0.006 |

*test for change over the three groups
^{†}p < 0.05 versus controls
^{‡}p < 0.001 versus controls

TABLE 11
Clinical pulmonary edema score

| Sum of | Score |
|---|---|
| Clinical edema score | |
| No lung crepitations, no 3rd heart sound | 1 |
| Crepitations (<50%) OR 3rd heart sound | 2 |
| Crepitations >50% lung field) | 3 |
| Acute pulmonary edema | 4 |
| (dyspnea, tachycardia, tachypnea, | |
| hypertension, diaphoresis, crepitations | |
| [>50%], and respiratory failure) | |
| And | |
| Orthopnea score | |
| None | 0 |
| Possible | 1 |
| Definite | 2 |
| | Total |

TABLE 12
Patient characteristics

| Etiology of heart disease | |
|---|---|
| Ischemic | 17 |
| Valvular | 2 |
| Idiopathic | 8 |
| Alcohol | 1 |
| Premorbid EF, % | 28 ± 1.9 |
| Premorbid NYHA class | II: 15 (54%) |
| | III: 13 (46%) |
| Time from symptom onset to presentation, hrs | 4.3 ± 0.6 |
| Maximum CK-MB, μg/L | 9.5 (2, 33)^{a} |
| Length of hospitalization, days | 6.5 ± 0.3 |

EF, left ventricular ejection fraction;
NYHA, New York Heart Association;
CK-MB, creatine kinase MB isoform.
^{a}Median (25th, 75th percentile).

TABLE 13
Markers of pulmonary edema after treatment

| | Presentation | Posttreatment |
|---|---|---|
| 1 hr | | |
| Systolic blood pressure, mm Hg | 168 ± 5 | 116 ± 3a |
| Diastolic blood pressure, mm Hg | 98 ± 4 | 73 ± 2a |
| Heart rate, beats/min | 128 ± 5 | 94 ± 4a |
| <2 hrs | | |
| PaO₂/FIO₂ | 190 ± 17 | 327 ± 23^{a} |
| 3 days | | |
| Chest radiograph EVLW score | 48 ± 3 | 11 ± 2a |

EVLW, extra vascular lung water.
^{a}p < .001.

### BIBLIOGRAPHY

Anderson B. Echocardiography. The normal examination and echocardiographic measurements. 1^{st} Edition, 2000, MGA Graphics, Manly, Australia, p 140.
Bersten A D, Holt A W, Vedig A E, et al: Treatment of severe cardiogenic pulmonary edema with continuous positive airway pressure delivered by facemask. N Engl J Med 1991; 325:1825-1830
Brown Norway rat model of allergic pulmonary inflammation. J Immunol Methods 1996;198:1-14
Chin M H. Heart failure outcomes in the community: Clinical and policy implications for a vulnerable population. Am J Med 1999;107:634-6
Davidson K G, Bersten A D, Barr H A et al. Lung function, permeability, and surfactant composition in oleic acid-induced acute lung injury in rats. Am J Physiol—Lung Cell Mol Physiol 2000; 279:L1091-102
Davies, S. W., Gailey, J., Keegan, J., Balcon, R., Rudd, R. M., Lipkin, D. L., Reduced pulmonary vascular permeability in severe chronic left heart failure. Am Heart J 124:137-142, 1992
Doyle I R, Bersten A D, Nicholas T E. Surfactant proteins-A and -B are elevated in plasma of patients with acute respiratory failure. Am J Respir Crit Care Med. 1997;156:1217-29
Doyle I R, Bersten A D, Nicholas T E: Surfactant proteins-A and -B are elevated in plasma of patients with acute respiratory failure. Am J Respir Crit Care Med 1997; 156: 1217-1229
Doyle I R, Nicholas T E, Bersten A D. Serum surfactant protein-A levels in patients with acute cardiogenic pulmonary edema and adult respiratory distress syndrome. Am J Respir Crit Care Med 1995;152: 307-17
Doyle I R, Nicholas T E, Bersten A D: Serum surfactant protein-A levels in patients with acute cardiogenic pulmonary edema and adult respiratory distress syndrome. Am J Respir Crit Care Med 1995; 152:307-317
Feigenbaum H. Echocardiography. 5^{th} Edition, 1994, Lea and Febiger, Philadelphia, USA, p 201-6.
Feinstein A R, Fisher M B, Pigeon J G. Changes in dyspnea-fatigue ratings as indicators of quality of life in the treatment of congestive heart failure. Am J Cardiol 1989;64:50-5
Gibbons R J et al. ACC/AHA/ACP-ASIM Guidelines for the Management of Patients with Chronic Stable Angina: A report of the American College of Cardiology/American Heart Association Task Force on Practice Guidelines (Committee on Management of Patients with Chronic Stable Angina). J Am Coll Cardiol 1999;33:2092-197
Givertz M M, Collucci W S, Braunwald E. Clinical aspects of heart failure: High-output failure; Pulmonary edema. In: Braunwald E, ed. Heart Disease. A textbook if cardiovascular medicine. Philadelphia: WB Saunders Co; 2001:534-57
Guazzi, J., Alveolar-capillary membrane dysfunction in chronic heart failure: pathophysiology and therapeutic implications. Clin Sci 98:633-641, 2000
Haites N E, McLennan F M, Mowat D H et al. How far is the cardiac output? Lancet 1984;2:1025-7
Heard, B. E., Path, F. C., Steiner, R. E., Herdan, A., Gleason, D., Oedema and fibrosis of the lungs in left ventricular failure. Br J Radiol 41:161-171, 1968;
Huang, W., Kingsbury, M. P., Turner, M. A., Donnelly, J. L., Flores, N. A., Sheridan, D. J., Capillary filtration is reduced in lungs adapted to chronic heart failure: morphological and haemodynamic correlates. Cardiovascular Res 49:207-217, 2001
Kaplan, J. D., Calandrino, F. S., Schuster, D. P., A positron emission tomographic comparison of pulmonary vascular permeability during the adult respiratory distress syndrome and pneumonia. Am Rev Respir Dis 143:150-154, 1991
Kay, J. M., Edwards, F. R., Ultrastructure of the alveolar-capillary wall in mitral stenosis. J. Pathol. 111:239-245, 1973
Killip T, Kimball J T: Treatment of myocardial infarction in a coronary care unit: A two-year experience with 250 patients. Am J Cardiol 1967; 20:457-464
Krum H. Reducing the burden of chronic heart failure. Med J Aust 1997;167:61-2
Kuroki Y, Tsutahara S, Shijubo N et al. Elevated levels of lung surfactant protein A in sera from patients with idiopathic pulmonary fibrosis and pulmonary alveolar proteinosis. Am Rev Respir Dis 1993; 147:723-9
Lee Y-S. Electron microscopic studies of the alveolar-capillary barrier in the patients of chronic pulmonary edema. Jpn Circ J 1979;43:945-54
Mahler D A, Weinberg D H, Wells C K et al. The measurement of dyspnea. Contents, interobserver agreement, and physiologic correlates of two new clinical indexes. Chest 1984;85:751-8.
McMurray J, Hart W. The economic impact of heart failure on the UK National Health Service. Eur Heart J 1993;14:133
Nicholas T E. Control of turnover of alveolar surfactant. NIPS 1993;8: 12-8
Oliver J R, Twidale N, Lakin C et al. Plasma atrial natriuretic polypeptide concentrations during and after reversion of paroxysmal supraventricular tachycardias. Br Heart J 1988;59:458-62
Pfeffer M A, Pfeffer J M, Fishbein M C et al. Myocardial infarct size and ventricular function in rats. Circ Res 44:503-512,1979
Pistolesi M, Giuntini C: Assessment of extravascular lung water. Radiol Clin North Am 1978; 16:551-574
Schiller N B, Shah P M, Crawford M et al. Recommendations for quantification of the left ventricle by two-dimensional echocardiography: American Society of Echocardiography Committee on Standards, Subcommittee on Quantification of Two-Dimensional Echocardiograms. J Am Soc Echocardiogr 1989;2:358-367
Schneider T. Issekutz A C. Quantitation of eosinophil and neutrophil infiltration into rat lung by specific assays for eosinophil peroxidase and myeloperoxidase. Application in a
Senni M, Tribouilloy C M Rodeheffer R J et al. Congestive heart failure in the community: a study of all incident cases in Olmsted County, Minn., in 1991. Circulation 1998;98:2282-9
Townsley, M. I., Fu, Z., Mathieu-Costello, O., West, J. B., Pulmonary microvascular permeability. Responses to high vascular pressure after induction of pacing-induced heart failure in dogs. Circ Res 77:317-325, 1995
Wallenstein S, Zucker C L, Fleiss J L. Some statistical methods useful in circulation research. Circ Res 1980;47:1-9
Wallenstein S, Zucker C L, Fleiss J L: Some statistical methods useful in circulation research. Circ Res 1980; 47:1-9
Yogalingam G, Doyle I R, Power J H T. Expression and distribution of surfactant proteins SP-A, SP-B, SP-C and lysozyme in the rat lung following prolonged periods of hyperpnea. Am J Physiol 1996;14:L320-L30

1. A method for detecting the onset or a predisposition to the onset of heart failure in a mammal, said method comprising screening for the modulation of systemic levels of pulmonary surfactant in said mammal wherein an increase in the level of pulmonary surfactant relative to normal levels is indicative of heart failure.

2. The method according to claim 1 wherein said detection is resolution of the onset or predisposition to the onset of heart failure in a mammal presenting with one or more non-exclusive symptoms or other indicators of heart failure.

3. The method according to claim 1 or 2 wherein said heart failure is congestive heart failure.

4. The method according to claim 3 wherein said congestive heart failure is acute or chronic heart failure.

5. The method according to any one of claims 1 to 4 wherein said systemic levels of pulmonary surfactant are the levels of pulmonary surfactant in a body fluid derived from said mammal.

6. The method according to claim 5 wherein said body fluid is blood or component thereof.

7. The method according to any one of claims 1 to 6 wherein said pulmonary surfactant is SP-A, SP-B, SP-C and/or SP-D.

8. The method according to claim 7 wherein said pulmonary surfactant is SP-B.

9. A method for monitoring the progression of heart failure in a mammal, said method comprising screening for the modulation of systemic levels of pulmonary surfactant in said mammal wherein the maintenance or increase in the level of pulmonary surfactant relative to a previously obtained surfactant level result in said mammal is indicative of the maintenance or worsening of said heart failure.

10. A method for monitoring the progression of heart failure in a mammal, said method comprising screening for the modulation of systemic levels of pulmonary surfactant in said mammal wherein a decrease in the level of pulmonary surfactant relative to a previously obtained surfactant level result in said mammal is indicative of an improvement in said heart failure.

11. The method according to claim 9 or 10 wherein said heart failure is congestive heart failure.

12. The method according to claim 11 wherein said congestive heart failure is acute or chronic heart failure.

13. The method according to any one of claims 9 to 12 wherein said systemic levels of pulmonary surfactant are the levels of pulmonary surfactant in a body fluid derived from said mammal.

14. The method according to claim 13 wherein said body fluid is blood or component thereof.

15. The method according to any one of claims 9 to 14 wherein said pulmonary surfactant is SP-A, SP-B, SP-C and/or SP-D.

16. The method according to claim 15 wherein said pulmonary surfactant is SP-B.

17. A method for assessing the severity of heart failure in a mammal, said method comprising quantitatively screening for the systemic level of pulmonary surfactant in said mammal wherein the degree of increase of said pulmonary surfactant level relative to normal levels is indicative of the severity of said heart failure.

18. The method according to claim 17 wherein said heart failure is congestive heart failure.

19. The method according to claim 18 wherein said congestive heart failure is acute or chronic heart failure.

20. The method according to any one of claims 17 to 19 wherein said systemic levels of pulmonary surfactant are the levels of pulmonary surfactant in a body fluid derived from said mammal.

21. The method according to claim 20 wherein said body fluid is blood or component thereof.

22. The method according to any one of claims 17 to 21 wherein said pulmonary surfactant is SP-A, SP-B, SP-C and/or SP-D.

23. The method according to claim 22 wherein said pulmonary surfactant is SP-B.

24. The method according to claim 23 wherein said SP-B level is predictive of the subsequent hospitalisation of said patient.

25. A method of detecting or monitoring for the onset of or a predisposition to the onset of a condition characterised by the presence of heart failure in a mammal, said method comprising screening for the modulation of systemic levels of pulmonary surfactant in said mammal wherein an increase in the level of pulmonary surfactant relative to normal levels is indicative of the onset or predisposition to the onset of said condition.

26. A method for monitoring a condition characterised by the presence of heart failure in a mammal said method comprising screening for the modulation of systemic levels of pulmonary surfactant in said mammal wherein an increase in the level of pulmonary surfactant relative to a previously obtained surfactant level result in said mammal is indicative of the maintenance or worsening of said condition.

27. A method for monitoring a condition characterised by the presence of heart failure in a mammal said method comprising screening for the modulation of systemic levels of pulmonary surfactant in said mammal wherein an decrease in the level of pulmonary surfactant relative to a previously obtained surfactant level result in said mammal is indicative of an improvement in said condition.

28. The method according to any one of claims 21 to 23 wherein said condition is abnormal heart valves, abnormally formed heart chambers, toxic or metabolic myocardial disease, hyperthyroidism, arrhythmia, dysrrhythmia, coronary artery disease, myocardial ischaemia, hypertension, myocarditis, severe lung disease or heart muscle damage.

29. A method for assessing the health status of a mammal said method comprising screening for the modulation of systemic levels of pulmonary surfactant in said mammal wherein an increase in the level of pulmonary surfactant relative to normal levels is indicative of heart failure.

30. The method according to any one of claims 25 to 29 wherein said heart failure is congestive heart failure.

31. The method according to claim 30 wherein said congestive heart failure is acute or chronic heart failure.

32. The method according to any one of claims 25 to 31 wherein said systemic levels of pulmonary surfactant are the levels of pulmonary surfactant in a body fluid derived from said mammal.

33. The method according to claim 32 wherein said body fluid is blood or component thereof.

34. The method according to any one of claims 25 to 33 wherein said pulmonary surfactant is SP-A, SP-B, SP-C and/or SP-D.

35. The method according to claim 34 wherein said pulmonary surfactant is SP-B.

36. The method according to any one of claims 1-35 wherein said mammal is a human.

37. A diagnostic kit for assaying biological samples said kit comprising in compartmental form a first compartment adapted to contain an agent for detecting pulmonary surfactant and a second compartment adapted to contain reagents useful for facilitating the detection by the agent in the first compartment.
